(19) United States
(12) Patent Application Publication
(10) Pub. No.: US 2012/0012123 A1
(43) Pub. Date: Jan. 19, 2012

(54) COMPOSITIONS, ARTICLES AND METHODS FOR PREVENTING OR REDUCING TOBACCO-ASSOCIATED DAMAGE

(75) Inventors: Moshe Gavish, Tel-Aviv (IL); Rafael M. Nagler, Timrat (IL)

(73) Assignee: Technion Research & Development Foundation Ltd., Haifa (IL)

(21) Appl. No.: 13/238,115

(22) Filed: Sep. 21, 2011

Related U.S. Application Data
(63) Continuation of application No. 12/588,825, filed on Oct. 29, 2009.
Application No. 12/588,825 is a continuation-in-part of application No. PCT/IL2008/000628, filed on May 6, 2008.
(60) Provisional application No. 60/924,272, filed on May 7, 2007.
(60) Provisional application No. 61/109,191, filed on Oct. 29, 2008.

(51) Int. Cl. A61K 31/197; A61K 9/68; A61P 35/00; A61P 9/10; A61P 9/00; A61P 11/00; A24B 15/32; A61K 8/44

(52) U.S. Cl. 131/334; 514/562; 424/48; 424/49

(57) ABSTRACT

Articles of manufacturing, methods, devices and compositions for preventing or reducing tobacco-associated damage in a subject, and which utilize a metal ion chelating agent, a copper chelating agent, a penicillamine and/or a structural analog of penicillamine, with and without an additional antioxidant, are disclosed.

### RELATED APPLICATIONS

This application is a continuation of U.S. patent application Ser. No. 12/588,825 filed on Oct. 29, 2009, which is a continuation-in-part (CIP) of PCT Patent Application No. PCT/IL2008/000628 filed on May 6, 2008, which claims the benefit of priority of U.S. Provisional Patent Application No. 60/924,272 filed on May 7, 2007.

U.S. patent application Ser. No. 12/588,825 also claims the benefit of priority of U.S. Provisional Patent Application No. 61/109,191 filed on Oct. 29, 2008.

The contents of all of the above applications are incorporated by reference as if fully set forth herein.

### FIELD AND BACKGROUND OF THE INVENTION

The present invention, in some embodiments thereof, relates to articles, compositions and methods for reducing or preventing tobacco-induced cellular and/or macromolecular damage.

The deleterious effects of tobacco abuse are well known. Tobacco is a worldwide public health hazard accounting for significant morbidity and mortality. Although smoking places an abundant oxidant insult to the oropharynx and respiratory tract, the oxidant burden associated with any tobacco consumption (as described hereinbelow) is deleterious to the entire body of the tobacco consumer.

Tobacco consumption leads to development or enhancement of atherosclerosis, cardiovascular diseases, chronic obstructive pulmonary disease, lung cancer, as well as other forms of cancer and peripheral vascular disease.

Cardiovascular disease is the main cause of death due to smoking. Cardiovascular disease can take many forms depending on which blood vessels are involved. Main forms include coronary thrombosis, which may lead to a heart attack; cerebral thrombosis, which may lead to collapse, stroke and paralysis; affected kidney arteries, which result in high blood pressure or kidney failure; and blockage of the vascular supply to the legs, which may lead to gangrene and amputation.

Tobacco consumers are more likely to get cancer than non-smokers, particularly carcinomas of the mouth, pharynx, esophagus and lung. Other types of cancers associated with tobacco consumption include bladder cancer, cancer of the oesophagus, cancer of the kidneys, cancer of the pancreas and cervical cancer.

Chronic obstructive pulmonary disease (COPD) is a collective term for a group of conditions that involve block of airflow and include, for example: emphysema and chronic bronchitis.

Other risks associated with tobacco consumption include hypertension, fertility problems, severe asthma, retinoic disorders such as macular degeneration and cataracts, ulcers, periodontal diseases, impotence, Diabetes type 2, Back pain, skin ailments such as premature ageing and wrinkling, osteoporosis, earlier menopause, and damaged and/or weakened immune system.

There are two principal ways to consume tobacco: smoking and smoke-less consumption. The latter comes in various forms: snuff, snus and chewing tobacco. Snuff is a fine-grain tobacco that often comes in teabag-like pouches, which users “pinch” or “dip” between their lower lip and gum. Chewing tobacco comes in shredded, twisted, or “bricked” tobacco leaves that users put between their cheek and gum. Whether it is snuff, snus or chewing tobacco, the user consumes the tobacco letting it sit in the mouth and suck on the tobacco juices, spitting often to get rid of the saliva that builds up. This sucking and chewing allows nicotine (a narcotic drug), to be absorbed into the bloodstream through the tissues of the mouth. Smokeless tobacco has a detrimental effect on the oral cavity plus systemic effects from buccal absorption of nicotine and other chemicals.

Evidence shows that cigars as well as cigarettes are highly toxic and addictive. Tobacco smokers have a similar increased risk for oral and laryngeal cancers. Evidence indicates that one cigar generates levels of carcinogenic particles exceeding those generated by three cigarettes. Fumes from cigars are also of greater consequence to secondary smokers. Epidemiologic studies reveal greater frequencies of heart disease, emphysema, and cancers of the mouth and pharynx in cigar smokers when compared to matched non-smokers.

Tobacco, whether smoked or chewed, causes common untoward effects in the oral cavity. Tobacco smoke has two chances to exert its deleterious effects in the mouth; when it is inhaled by the smoker and on its exit during exhalation.

Over 30,000 new cases of cancer of the oral cavity are diagnosed annually, accounting for 2-4 percents of all new cancers. The great majority of these patients are users of tobacco products.

Oral squamous cell carcinoma (SCC) is the most common malignancy of the head and neck with a worldwide incidence of over 300,000 new cases annually. The disease is characterized by a high rate of morbidity and mortality (approximately 50%) and in this respect is similar to malignant melanoma. The major inducer of oral SCC is exposure to tobacco which is considered to be responsible for 50-90% of cases world-wide [Epstein and Scully, SCD Special Care in Dentistry 1997; 17:120-8; Holleb et al. Textbook of Clinical Oncology. The American Cancer Society, 1991]. As such, the incidence of oral SCC in tobacco smokers is 4-7 times higher than in non-smokers [see, for example, Ko et al. J Oral Pathol Med 1995; 24:450-3].

Various malignancies are particularly associated with smokeless tobacco consumption. These include oral cancer and cancer of the gastrointestinal tract including esophagus and bladder. Leukoplakia, a tobacco induced white patch on the buccal mucosa, as found in smokers, is a localized irritation due to direct contact of smoked or smokeless tobacco and it is directly related to the frequency and years of tobacco abuse. Although leukoplakia is a benign oral lesion, it has a malignant potential.

In addition, tobacco contributes to other oral symptoms or pathologies of the mouth and teeth. Tobacco may cause halitosis, may numb the taste buds, and interfere with the smell and the taste of food. It may stain teeth and contribute to dental caries. Smokers have more dental tartar (calculus) than non-smokers. Tobacco is associated also with destructive periodontal (gum) disease and tooth loss. Acute necrotizing ulcerative gingivitis (“trench mouth”) is a destructive, painful inflammatory condition occurring mainly in tobacco smokers. Swelling of the nasal and sinus membranes has also been associated, purportedly, in individuals who are “allergic” to TS.

Oral submucous fibrosis occurs mainly in India and is a chronic, progressive premalignant condition. The etiology is chronic chewing of tobacco or areca nut or both. The fibrosis results in restriction of mouth opening and involves the palates, tonsillar fossa, buccal mucosa and underlying muscle. Associated with this condition are also oropharyngeal carcinomas, also with a high frequency in India and associated in 70% of cases with chewing tobacco. Smokeless tobacco and areca nut usage is also common in Pakistan, Bangladesh and Java and in these and Indian immigrants to the United States and United Kingdom.

Studies have estimated that TS has over 3,000 different constituents, of which many are toxic, carcinogenic and/or generate free radical species.

Free radicals are atoms or molecules containing an unpaired electron. Oxygen free radicals include the superoxide free radical (.O2⁻) and the hydroxyl radical (OH.) which, together with hydrogen peroxide (H₂O₂) and singlet oxygen (¹O2), are jointly called reactive oxygen species (ROS). Due to their high reactivity they may lead to chemical modification and impairment of the components of living cells, such as proteins, lipids, carbohydrates and nucleotides.

Tobacco smoke therefore induces oxidative damage to lipids, DNA and proteins, particularly via protein-SH groups as a consequence of containing high levels of both free radicals as well as aldehydes, including acetaldehyde (ethanol), propanol and acrolein, as well as other deleterious molecules.

Most of constituents of TS have been identified in so-called mainstream and side stream TS. The former is that volume of smoke drawn through the mouthpiece of the tobacco product during puffing while side stream smoke is that smoke emitted from the smoldering cigarette in between puffs. Although tar and nicotine are retained in the filter of cigarettes, this applies mainly to mainstream smoke, when comparing filter and non-filter cigarettes. Mainstream smoke emission is also markedly reduced both in low and in ultra low tar yield cigarettes. However, the emissions of toxic and carcinogenic components in side stream smoke are not significantly reduced in filter cigarettes when compared to non-filter counterparts. Thus, side stream smoke is a major contributor to environmental smoke, affecting both the smoker and their non-smoking counterparts, so called secondary smokers.

Tobacco smoke is divided into two phases; tar and gas-phase smoke. Tar contains high concentrations of free radicals. Many tar extracts and oxidants are water-soluble and reduce oxygen to superoxide radical which can dismutate to form the potent oxidant H₂O₂. Oxidants in gas-phase smoke are reactive carbon- and oxygen-centered radicals with extremely short half lives.

Cells subjected to oxidative stress develop severely affected cellular function and suffer damage to membrane lipids, to proteins, to cytoskeletal structures and to DNA. Free radical damage to DNA has been measured as formation of single-strand breaks, double-strand breaks and chromosomal aberrations. Cells exposed to ionizing radiation and TS have also been demonstrated to have an increased intracellular DNA damage, a precursor of mutations and development of malignancies. It has been shown that TS elicits protein carbonylation in plasma and that, in contrast, exposure of human plasma to gas-phase but not to whole TS produces oxidative damage to lipids.

Redox-active metal ions, such as iron and copper, in the presence of H₂O₂ and other low-reactive free radicals found in TS, such as superoxide radicals, participate in the deleterious Haber-Weiss and Fenton reactions, in which the highly reactive hydroxyl free radicals are produced.

Studies have shown that exposure of plasma to TS results in protein damage in the form of protein carbonylation [Reznick et al. Biochem J 1992; 286:607-11] and in oxidation of plasma lipids and antioxidants [Reznick et al. Redox Report 1997; 3:169-174]. The source of the accumulation of protein carbonylation was found to be due to aldehydes present in TS [O'Neill et al. J Lab Clin Med 1994; 124:359-370; Nagler et al. Arch Biochem Biophys 2000; 379:229-36]. In addition, it was shown that several salivary enzymes such as amylase, lactic dehydrogenase (LDH), and acid phosphatase were considerably affected by TS [Nagler et al., 2000, supra; Nagler et al. J Lab Clin Med 2001; 137:363-9], where both TS-based aldehydes, such as acrolein and crotonaldehyde, as well as oxygen free radicals were implicated as the causative agents affecting the above enzymes.

Glutathione, a sulfur-containing tripeptide (L-glutamyl-1-cysteine-glycine) is the most abundant non-protein thiol in mammalian cells and is recognized as the primordial antioxidant. Glutathione, in its reduced form, “GSH”, acts as a substrate for glutathione-S-transferase and glutathione peroxidase, enzymes catalyzing reactions involved in detoxification of xenobiotic compounds and in antioxidation of ROS and other free radicals. This ubiquitous protein plays a vital function in maintaining the integrity of free radical sensitive cellular components. Under states of GSH depletion, including malnutrition and severe oxidative stress, cells may then become injured from excess free radical damage and die.

Oral peroxidation is the pivotal enzymatic activity of the salivary antioxidant system. Oral peroxidase activity is composed of the combined activity of two peroxidases, salivary peroxidase (SPO) and myeloperoxidase (MPO). Salivary peroxidase, which is secreted by the major salivary glands, mainly the parotid gland, contributes 80% of the total oral peroxidase (OPO) activity, while MPO, produced by leukocytes, contributes the remaining 20% of OPO activity. Oral peroxidase performs two functions preventing oxidant injury; it reduces the level of H₂O₂ excreted into the oral cavity from the salivary glands, by bacteria and by leukocytes, and it inhibits the metabolism and proliferation of various bacteria in the oral cavity.

Oral peroxidase is involved in destroying TS-associated H₂O₂. Tobacco smoke-associated hydrocyanic acid (HCN) is metabolized by the liver to thiocyanate ion (SCN⁻). This SCN⁻ is specifically sequestered from the plasma by the parotid gland and is secreted by this gland into the oral cavity. Its concentration in the saliva of non-smokers ranges from 0.3-1.5 mM, while the respective range in smokers is approximately 1.4-4.0 mM, depending on the number of cigarettes smoked per day, with a prolonged t_{1/2} of 9.5 hours. Following its secretion in saliva, SCN⁻ reacts with, and eliminates H₂O₂ in the oral cavity in a reaction catalyzed by OPO, as described in FIG. 2a. However, it has been shown that if OPO is damaged or depleted, as occurs upon exposure to TS, the H₂O₂ in the oral cavity is not eliminated and remains available for further reaction with redox-active metal ions which are secreted via the parotid gland saliva.

In the following reaction: SCN⁻+H₂O₂→OSCN⁻+H₂O, which is catalyzed by OPO, H₂O₂ oxidizes SCN⁻, a detoxification product of cyanide secreted mainly by the parotid gland. In this reaction, SCN⁻ acts as the electron-donating component, similarly to GSH in other biological systems. Two potent antibacterial oxidizing products evolve from this reaction: hydrogen hypothiocyanite (HOSCN) and its conjugated anion, OSCN⁻. The antibacterial activity of HOSCN and OSCN⁻ stems from their ability to react with sulfhydryl groups of bacterial enzymes that are vital for glycolysis, such as hexokinase, aldolase and pyruvate kinase.

The importance of OPO in oral disease prevention has been demonstrated in several studies. For example, studies using animal models or the Ames test have shown that saliva inhibits the mutagenicity of known oral cancer inducers, such as TS, 4NQO and benzopyrene. Biochemical studies have also demonstrated that saliva inhibits production of ROS such as superoxide free radical and H₂O₂ from betel quid tobacco, the most potent inducer of oral cancer. These observations are further supported in the observation that patients with oral lichen planus, a premalignant lesion, have reduced salivary antioxidant capacity.

Several prior art approaches have been employed in order to reduce or prevent incidence of oral disease resulting from oxidant injury.

For example, cigarette filters are used to trap TS tar but do not affect the gas-phase compounds.

One approach has employed a filter for TS providing chemoabsorptive properties to reduce aldehyde concentration in TS (see, U.S. Pat. No. 5,060,672).

Another approach has employed oral megadoses of antioxidants in attempts to reduce generation of H₂O₂ resulting from the “respiratory burst” reaction associated with phagocytic activity of macrophages and neutrophils. It has been shown that smokers have a higher “respiratory burst” reaction than non-smokers and that this may be associated with the increased incidence of aerodigestive tract disease in the former.

In yet another approach, dipeptide compounds with pharmaceutical properties to increase glutathione levels were employed (see, for example, U.S. Pat. No. 4,761,399).

A further approach utilized a glycine carboxylic acid alkyl mono-ester of glutathione to increase cellular GSH levels (see, for example, U.S. Pat. No. 4,710,489).

In yet a further approach, administration of a combination of glutathione and selenium was suggested for preventing oxidant injury resulting from exposure to TS (see, for example, U.S. Pat. No. 5,922,346).

In another approach, administration of a combination of glutathione, ascorbic acid, selenium and a sulfur-containing amino acid was suggested in order to prevent oral oxidant injury (see, for example, U.S. Pat. No. 6,228,347).

In yet another approach, administration of a combination including some or all of the following antioxidants; L-glutathione, L-selenomethionine, L-selenocysteine, ascorbyl palmitate, ascorbic acid esters, L-cysteine, N-acetyl-1-cysteine, tocopherol acetate, tocopherol succinate, vitamin A, a zinc salt, methionine and taurine was suggested in order to provide intra-oral protection from oxidant injury (see, U.S. Pat. No. 5,829,449)

The present inventors have previously described novel smoking filters and oral compositions for reducing tobacco associated damage in the aerodigestive tract (see, U.S. Pat. No. 6,789,546, which is incorporated by reference as if fully set forth herein). These compositions include active agents which are capable of reducing or preventing tobacco associated loss of peroxidase activity in the aerodigestive tract.

U.S. Pat. No. 5,922,346 teaches a composition for reducing free radical damage induced by tobacco products and environmental pollutants comprising, as active ingredients, reduced glutathione and a source of selenium selected from the group consisting of elemental selenium, selenomethionine and selenocysteine, the active ingredients being combined with suitable carriers and flavorings for their intra-oral administration as gels, lozenges, tablets and gums in concentrations for reducing free radical damage induced by tobacco products and other environmental pollutants to the oral cavity, pharynx and upper respiratory tract of a user and secondary smokers.

U.S. Pat. No. 5,906,811 teaches a method for reducing free radical damage induced by tobacco products and environmental pollutants comprising administering in a suitable carrier in concentrations for effectively reducing said free radical damage to the oro-pharynx and upper respiratory tract of a user a combination of from 0.01 and 10% (weight) glutathione, from 1.0 to 25% (weight) ascorbic acid, from 0.001 to 10% (weight) of a source of selenium and from 0.001 to 2.0% (weight) of a sulfur containing amino acid.

These aforementioned attempts to reduce tobacco damage are used as an adjuvant treatment following or prior to tobacco consumption, but not concomitantly with tobacco consumption.

U.S. Pat. No. 5,829,449 teach a composition for inclusion within a cigarette, cigar or pipe tobacco for reducing free radical damage to the oro-pharyngeal cavity, respiratory tract and lungs from tobacco smoke, said composition comprising L-glutathione and a source of selenium selected from the group consisting of L-selenomethionine and L-selenocysteine. U.S. Pat. No. 5,829,449 clearly states that the composition is supplied by smoke inhalation and not by direct contact with the aerodigestive tract (i.e., wet tissue).

U.S. Pat. No. 6,138,683 teaches a composition for inclusion within a quantity of smokeless tobacco, selected from the group consisting of chewing tobacco and snuff, for reducing free radical induced damage to the oro-pharyngeal cavity of the user, said composition comprising L-glutathione and a source of selenium in combination with said smokeless tobacco.

PCT/IL2008/000101, by the present assignee, describes methods, pharmaceutical compositions, oral compositions, filters and tobacco products for preventing or reducing tobacco smoke-associated injury in the aerodigestive tract of a subject, which can be used to prevent or reduce loss of OPO activity or CN⁻-, redox-active metal ion- or aldehyde-induced cell death resulting from TS-associated oxidative stress. Some of the agents described in this document are CN⁻ chelators and iron chelators.

D-Penicillamine (see, FIGS. 1A and 1B), is a known compound often considered as a cysteine analog and is further known, inter alia, as a copper chelator (See, FIG. 1B).

The following background art describes some of the recently disclosed features of D-penicillamine:

Handel et al., Clinical and Experimental Pharmacology and Physiology (2000) 27, 139-144; M. L. Handel, Inflamm. res. 46 (1997) 282-286; and P. E. Lipsky, J. Clin. Invest. 73 (1984), 53-65.

Additional related art include Fugioka et al., Molecula and cellular biology, September 2004, p. 7806-7819; Bar-Shai et al., Journal of Physiology and pharmacology 2006, 57, Supp 4, 39.44; Birrell et al., Journal of cellular biology (2007) pp. 27-37; Font et al., Psychopharmacology 2006 184(1):56-64; Wood et al., Eur J Pharmacol. 2008 580(1-2):48-54; et al. J Endourol. 2005 April; 19(3):429-32; and Munro and Capell, Br J Rheumatol. 1997 January; 36(1):104-9.

### SUMMARY OF THE INVENTION

The prior art fails to teach or suggest a role for penicillamine, as well as copper and other metal ion chelating agents in general, in the treatment of tobacco-associated damage.

The present inventors have now shown that penicillamine beneficially affects tobacco-associated cellular and macromolecular damage induced by exposure to tobacco smoke, even in the absence of saliva, and thus can serve as a potent agent for treating tobacco-associated damage. Similar effects are exhibited by copper chelating agents and chelating agents of other redox active metals.

According to an aspect of some embodiments of the present invention there is provided an article of manufacturing comprising tobacco and a tobacco packaging material, wherein at least a portion of the tobacco and/or tobacco packaging material comprises an agent selected from the group consisting of penicillamine and a structural analog of penicillamine.

According to an aspect of some embodiments of the present invention there is provided an article of manufacturing comprising tobacco and an agent being incorporated in at least a portion of the tobacco, the agent being selected from the group consisting of penicillamine and a structural analog of penicillamine.

According to an aspect of some embodiments of the present invention there is provided an article of manufacturing comprising a tobacco packaging material and an agent being incorporated in at least a portion of the tobacco packaging material, the agent being selected from the group consisting of penicillamine and a structural analog of penicillamine.

According to an aspect of some embodiments of the present invention there is provided an article of manufacturing comprising tobacco and a tobacco packaging material, wherein at least a portion of the tobacco and/or tobacco packaging material comprises an agent, the agent being a metal ion chelating agent, wherein the metal is such that can assume two or more oxidation states other than 0.

According to an aspect of some embodiments of the present invention there is provided an article of manufacturing comprising tobacco and an agent being incorporated in at least a portion of the tobacco, the agent being a metal ion chelating agent, wherein the metal is such that can assume two or more oxidation states other than 0.

According to an aspect of some embodiments of the present invention there is provided an article of manufacturing comprising a tobacco packaging material and an agent being incorporated in at least a portion of the tobacco packaging material, the agent being a metal ion chelating agent, wherein the metal is such that can assume two or more oxidation states other than 0.

According to some embodiments of the invention, at least a portion of the tobacco and/or the tobacco packaging material is in contact with an aerodigestive tract of a subject using the article of manufacturing.

In some embodiments, the agent is penicillamine.

In some embodiments, the agent is D-penicillamine.

According to some embodiments of the invention, the structural analog of penicillamine has the general formula:

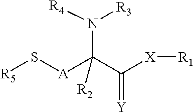

wherein:

X is O or NR₆;

Y is O;

A is CR₇R₈ or CR₇R₈—CR₉R₁₀;

R₁ is selected from the group consisting of hydrogen, a substituted or unsubstituted alkyl, a substituted or unsubstituted cycloalkyl and a substituted or unsubstituted aryl, whereas when anyone of the alkyl, aryl and the cycloalkyl is substituted, the substituent is independently selected from the group consisting of alkyl, cycloalkyl, alkoxy, aryl and aryloxy;

R₂ is hydrogen or alkyl;

R₃ and R₄ are each independently selected from the group consisting of hydrogen, alkyl, alkenyl, cycloalkyl and aryl, or, alternatively, R₃ and R₄ are linked therebetween so as to form a five- or six-membered nitrogen-containing heteroalicyclic ring, or, alternatively, at least one of R₃ and R₄ forms a five- or six-membered heteroalicyclic ring with R₅;

R₅ is selected from the group consisting of hydrogen, a substituted or unsubstituted alkyl, a substituted or unsubstituted cycloalkyl and a substituted or unsubstituted aryl, whereas when anyone of the alkyl, the cycloalkyl and the aryl is substituted, the substituent is independently selected from the group consisting alkyl, cycloalkyl, alkoxy, aryl, aryloxy, carbonyl, aldehyde and carboxy, or, alternatively, R₅ forms a five- or six-membered heteroalicyclic ring with one of R₃ and R₄;

R₆ is selected from the group consisting of hydrogen, a substituted or unsubstituted alkyl, a substituted or unsubstituted cycloalkyl and a substituted or unsubstituted aryl, whereas when anyone of the alkyl, the aryl and the cycloalkyl are substituted, the substituent is independently selected from the group consisting of alkyl and cycloalkyl, or, alternatively, R₆ forms with R₁ a nitrogen-containing five-, six- or seven-membered ring; and

R₇, R₈, R₉ and R₁₀ are each independently selected from the group consisting of hydrogen and alkyl.

In some embodiments, the penicillamine or the structural analog thereof is capable of suppressing an innate immune activity in a subject using the article of manufacturing.

In some embodiments, the penicillamine or the structural analog thereof is capable of inhibiting inflammation in a subject using the article of manufacturing.

According to some embodiments of the invention, the agent is capable of reducing or preventing tobacco smoke-associated damage in a subject using the article of manufacturing.

In some embodiments, the metal that can assume two or more oxidations states other than 0 is a redox active metal selected from the group consisting of iron, copper and nickel. In some embodiments, the redox active metal is copper.

In some embodiments, the metal ion chelating agent is selected from the group consisting of penicillamine, trientine, ethylendiamine, diethylenetriamine, triethylenetetramine, triethylenediamine, aminoethylethanolamine, aminoethylpiperazine, pentaethylenehexamine, triethylenetetramine, captopril, N,N′-bis(3-aminopropyl)-1,3-propanediamine, N,N′-Bis(2-aminoethyl)-1,3-propanediamine, 1,7-dioxa-4,10-diazacyclododecane, 1,4,8,11-tetraazacyclotetradecane-5,7-dione, 1,4,7-triazacyclononane, 1-oxa-4,7,10-triazacyclododecane, 1,4,8,12-tetraazacyclopentadecane, 1,4,7,10-tetraazacyclododecane, staurosporine aglycone, 4,5-dianilinophthalimide, 1,10-phenanthroline, 1,2-diaminobenzene, and derivatives or structural analogs of any of the above.

In some embodiments the metal ion chelating agent is a linear or cyclic polyamine.

According to some embodiments of the invention, the at least a portion of the tobacco and/or the tobacco packaging material further comprises at least one additional agent capable of reducing or preventing tobacco smoke-associated damage in a subject using the article of manufacturing.

According to some embodiments of the invention, the additional agent is selected from the group consisting of an antioxidant, an iron chelating agent, a cyanide chelating agent and an agent capable of reducing or preventing tobacco associated loss of peroxidase activity in an aerodigestive tract of the subject.

In some embodiments, the agent is desferal.

According to some embodiments of the invention, the tobacco packaging material comprises a filter and the agent is impregnated in a paper of the filter.

According to some embodiments of the invention, the tobacco is smokeless tobacco.

According to some embodiments of the invention, the tobacco is smoked tobacco.

According to some embodiments of the invention, the tobacco packaging material is selected from the group consisting of a rolling paper, a filter paper, a snus bag packaging, a cigarette, a pipe and a tin sheet packaging.

According to some embodiments of the invention, the article of manufacturing is selected from the group consisting of a snuff, a cigarette, a snus, a Gutka, a plug, a twist, a scrap and tobacco water.

According to an aspect of some embodiments of the present invention there is provided a method of treating or preventing a tobacco-associated damage in a subject in need thereof, the method comprising administering to the subject a therapeutically effective amount of an agent selected from the group consisting of penicillamine and a structural analog thereof.

According to an aspect of some embodiments of the present invention there is provided use of an agent selected from the group consisting of penicillamine and a structural analog thereof in the manufacture of a medicament for treating or preventing a tobacco-associated damage.

According to an aspect of some embodiments of the present invention there is provided a pharmaceutical composition comprising an agent selected from the group consisting of penicillamine and a structural analog thereof and a pharmaceutically acceptable carrier, the composition being packaged in a packaging material and identified in print, in or on the packaging material, for use in the treatment of a tobacco-associated damage.

According to an aspect of some embodiments of the present invention there is provided a method of treating or preventing a tobacco-associated damage in a subject in need thereof, the method comprising administering to the subject a therapeutically effective amount of an agent, the agent being a metal ion chelating agent, wherein the metal is such that can assume two or more oxidation states other than 0.

According to an aspect of some embodiments of the present invention there is provided use of an agent being a metal ion chelating agent in the manufacture of a medicament for treating or preventing a tobacco-associated damage, wherein the metal is such that can assume two or more oxidation states other than 0.

According to an aspect of some embodiments of the present invention there is provided a pharmaceutical composition comprising an agent and a pharmaceutically acceptable carrier, the agent being a metal ion chelating agent, the composition being packaged in a packaging material and identified in print, in or on the packaging material, for use in the treatment of a tobacco-associated damage, wherein the metal is such that can assume two or more oxidation states other than 0.

According to some embodiments of the invention, the tobacco-associated damage is effected in a mucosal tissue.

According to some embodiments of the invention, the tobacco-associated damage is effected in a non-mucosal tissue.

According to some embodiments of the invention, the agent is D-penicillamine.

According to some embodiments of the invention, the structural analog of penicillamine has the general formula described hereinabove.

According to some embodiments of the invention, the metal ion chelating agent is as described hereinabove.

According to some embodiments of the invention, the agent is used in combination with at least one additional agent that is capable of reducing or preventing the tobacco-associated damage.

According to some embodiments of the invention, the additional agent is an antioxidant.

In some embodiments, the antioxidant is desferal.

According to an aspect of some embodiments of the present invention there is provided an article of manufacturing comprising a filter and an agent comprised with the filter, the agent being selected from the group consisting of penicillamine and a structural analog of penicillamine, as described herein, and the filter being designed and configured so as to enable release of the agent therefrom when in use by a subject.

According to an aspect of some embodiments of the present invention there is provided an article of manufacturing comprising a filter and an agent comprised with the filter, the agent being a metal ion chelating agent, as described herein, and the filter being designed and configured so as to enable release of the agent therefrom when in use by a subject.

According to an aspect of some embodiments of the present invention there is provided an oral composition comprising an agent selected from the group consisting of penicillamine and a structural analog of penicillamine, as described herein, the composition being in the form of a toothpaste, powder, liquid dentifrice, mouthwash, denture cleanser, chewing gum, lozenge, paste, gel or candy.

According to an aspect of some embodiments of the present invention there is provided an oral composition comprising a metal ion chelating agent, as described herein, the composition being in the form of a toothpaste, powder, liquid dentifrice, mouthwash, denture cleanser, chewing gum, lozenge, paste, gel or candy.

In some embodiments, the oral composition further comprises a flavorant.

According to an aspect of some embodiments of the present invention there is provided a medical device comprising an agent selected from the group consisting of penicillamine and a structural analog of penicillamine, as described herein, the medical device being designed and configured to deliver the agent to a bodily site.

According to an aspect of some embodiments of the present invention there is provided a medical device comprising a metal ion chelating agent, as described herein, the medical device being designed and configured to deliver the agent to a bodily site.

In some embodiments, the medical device is for delivering the agent by topical or transdermal application.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present the 2-D chemical structure of D-penicillamine (FIG. 1A) and of Penicillamine as a copper chelator, in which one atom of copper is combined with two molecules of PenA (FIG. 1B).

FIG. 2 is schematic diagram depicting the construction of a filter paper impregnated with agents according to embodiments of the present invention.

FIG. 3 are comparative plots showing the survival rate of H1299 cells incubated in the presence of medium+saliva and exposed to CS (♦); of H1299 cells incubated in the presence of medium alone and exposed to CS (▪); of H1299 cells incubated in the presence of medium+saliva (▴); and of H1299 cells incubated in the presence of medium (); (n=3), and demonstrating the synergistic effect of CS and saliva on cells survival.

FIG. 4 is a bar graph presenting the survival rates of H1299 cells incubated in the presence of medium+saliva and exposed to CS (rhomb); of H1299 cells incubated in the presence of medium alone and exposed to CS (inlaid); of H1299 cells incubated in the presence of medium+saliva (stripe); and of H1299 cells incubated in the presence of medium (diagonal stripe), at 120 minutes; *P=0.005 (n=11), and further demonstrating the synergistic effect of CS and saliva on cell survival at this time point.

FIG. 5A presents comparative plots showing the survival rate of H1299 cells incubated in the presence of medium+saliva+5 mM PenA and exposed to CS (brown circles); of H1299 cells incubated in the presence of medium+5 mM PenA and exposed to CS (purple stars); of H1299 cells incubated in the presence of medium alone and exposed to CS (pink squares); of H1299 cells incubated in the presence of medium+saliva (yellow triangles); of H1299 cells incubated in the presence of medium+saliva and exposed to CS (blue diamonds); and of H1299 cells incubated in the presence of medium (turquoise) (n=3), and demonstrating the effect of PenA on cells survival.

FIG. 5B is a bar graph presenting the survival rate of H1299 cells incubated under the condition described herein for FIG. 5a, at 120 minutes, with SDs.

FIGS. 6A and 6B are bar graphs presenting the survival rate of H1299 cells incubated in the presence of medium+saliva+5 mM PenA and exposed to CS; of H1299 cells incubated in the presence of medium+5 mM PenA and exposed to CS; of H1299 cells incubated in the presence of medium alone and exposed to CS; of H1299 cells incubated in the presence of medium+saliva; of H1299 cells incubated in the presence of medium+saliva and exposed to CS; and of H1299 cells incubated in the presence of medium, as the percent of viable cells out of the control group (FIG. 6A) and out of the total number of cells (FIG. 6B).

FIG. 7 presents bar graphs showing the effect of 1 mM (middle bars) and 5 mM (right bars) PenA treatment, compared to non-treatment (left bars) on survival of H1299 lung cancer incubated in the presence of medium+30% saliva and exposed to CS (rhomb); of H1299 cells incubated in the presence of medium alone and exposed to CS (inlaid); of H1299 cells incubated in the presence of medium+30% saliva (stripe); and of H1299 cells incubated in the presence of medium (diagonal stripe). Samples were also incubated in the presence of 1 mM or 5 mM PenA prior the exposure of CS (n=7). *p<0.001,**p<0.001.

FIG. 8 presents data plots depicting the effects of 5 mM Penicillamine (PenA) on survival of H1299 lung cancer cells incubated in the presence of medium+saliva (A); of H1299 cells incubated in the presence of medium alone (B); of H1299 cells incubated in the presence of medium+saliva and 5 mM PenA (C); of H1299 cells incubated in the presence of medium with 5 mM PenA (D); of H1299 cells incubated in the presence of medium+saliva and exposed to CS (E); of H1299 cells incubated in the presence of medium alone and exposed to CS (F); of H1299 cells incubated in the presence of medium+saliva and 5 mM PenA and exposed to CS (G); and of H1299 cells incubated in the presence of medium with 5 mM PenA and exposed to CS(H). Cells were exposed to CS for 120 minutes, samples were incubated with/without 5 mM PenA prior the exposure to CS, and re-suspended with 10 μg/ml PI for 10 minutes.

FIG. 9 presents bar graphs showing the effect of 2 mM GSH treatment (left bars), compared to non-treated control (left bars) on the survival of H1299 human lung cancer cells incubated in the presence of medium+30% saliva and exposed to CS (rhomb); of H1299 cells incubated in the presence of medium alone and exposed to CS (inlaid); of H1299 cells incubated in the presence of medium+30% saliva (stripe); and of H1299 cells incubated in the presence of medium (diagonal stripe). Samples were also incubated in the presence of 2 mM GSH prior the exposure of CS (n=3).

FIG. 10 are comparative plots showing the protein carbonylation level of H1299 cells incubated in the presence of medium+saliva and exposed to CS (♦); of H1299 cells incubated in the presence of medium alone and exposed to CS (▪); of H1299 cells incubated in the presence of medium supplement with 5 mM PenA+saliva and exposed to CS (▴); and of H1299 cells incubated in the presence of medium supplement with 5 mM PenA and exposed to CS () (n=3).

FIG. 11 are bar graphs showing the effect of 5 mM PenA treatment (right bars), compared with non-treated control (left bars) on protein carbonylation level of H1299 human lung cancer cells incubated in the presence of medium+saliva and exposed to CS during 60 minutes (rhomb); of H1299 cells incubated in the presence of medium alone and exposed to 60 minutes CS (inlaid). Samples were also incubated in the presence of 5 mM PenA prior the exposure of CS (n=3).

FIG. 12 presents bar graphs showing the effect of 5 mM Desferal treatment (right bars), compared to non-treated control (left bars) on the survival of H1299 human lung cancer cells incubated in the presence of medium+30% saliva and exposed to CS (rhomb); of H1299 cells incubated in the presence of medium alone and exposed to CS (inlaid); of H1299 cells incubated in the presence of medium+30% saliva (stripe); and of H1299 cells incubated in the presence of medium (diagonal stripe). Samples were also incubated in the presence of 5 mM Desferal prior the exposure of CS (n=3).

FIG. 13 presents bar graphs showing the synergistic effect of 5 mM Desferal and 5 mM PenA on the survival of H1299 human lung cancer cells treated as described herein.

FIG. 14 presents bar graphs showing the CS induced changes in mitochondrial membrane potential without treatment (left bars), in the presence of PenA (middle bars) and in the presence of DES (right bars). H1299 cells incubated in the presence of medium+saliva and exposed to CS (rhomb). H1299 cells incubated in the presence of medium alone and exposed to CS (inlaid). Samples were also incubated in the presence of 5 mM PenA or 5 mM DES prior the exposure of CS. Cells were incubated for 30 minutes with JC-1 followed by exposure to CS for 120 minutes.

FIG. 15 presents bar graphs showing that CS leads to mitochondrial membrane damage. H1299 cells incubated in the presence of medium+saliva and exposed to CS (rhomb). H1299 cells incubated in the presence of medium alone and exposed to CS (inlaid). Samples were also incubated in the presence of 5 mM PenA or 5 mM DES prior the exposure of CS. Cells were incubated for 30 minutes with NAO followed by exposure of CS for 120 minutes (n=3).

FIG. 16 presents the Western blot analysis showing the effect of 5 mM PenA on the protein levels of total p53 following CS exposure and saliva supplementation in H1299 cells. Total protein (20 μg) were loaded onto gradient SDS gels and transferred to nitrocellulose.

FIG. 17 presents the Western blot analysis showing the effect of 2 mM GSH on the protein levels of total p53 following CS exposure and saliva supplementation. Total protein (20 μg) were loaded onto gradient SDS gels and transferred to nitrocellulose.

FIGS. 18A-B present bar graphs presenting the survival rates of oral cancer cells in the absence (FIG. 18A) and presence (FIG. 18B) of saliva, as measured by the Trypan blue assay, upon incubating the cells for 120 minutes in medium with/without 5 mM PenA and upon exposure (or absence thereof) to CS. **p<0.01 depicts statistical significant difference in the survival rate of cells protected by the PenA and cells which did not survive. In FIG. 18A, (n=14) and (p=0.0024) and in FIG. 1B, (n=7) and (p=0.015).

### DETAILED DESCRIPTION OF EMBODIMENTS OF THE INVENTION

The present invention is of articles, methods and compositions for preventing or reducing tobacco smoke-associated damage. Specifically, the present invention, in some embodiments thereof, is of methods, pharmaceutical compositions, oral compositions, medical devices, filters and tobacco products, which are useful in preventing or reducing tobacco smoke-associated damage, and which utilize penicillamine, structural analogs thereof and/or other chelating agents of redox-active metals such as copper.

The principles and operation of some embodiments of the present invention may be better understood with reference to the drawings and accompanying examples.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Tobacco consumption, such as in the form of smoking, chewing, dipping or snuffing, is associated with pathogenesis of many diseases.

The present inventors have previously described novel smoking filters and oral compositions for reducing tobacco associated damage in the aerodigestive tract (see, U.S. Pat. No. 6,789,546). These compositions include active agents which are capable of reducing or preventing tobacco associated loss of peroxidase activity in the aerodigestive tract. Some of the present inventors have previously taught tobacco compositions and tobacco packaging means that prevent or reduce loss of OPO activity or CN⁻, redox-active metal ion- or aldehyde-induced cell death resulting from TS-associated oxidative stress (see, PCT/IL2008/000101). While these disclosures teach agents that exert their activity via contacting saliva, agents that are capable of reducing or preventing tobacco-associated damage, with and without the presence of saliva, have not been taught heretofore.

As discussed hereinabove, tobacco consumption may lead to various, severe tobacco-associated damage, which affects millions subjects every year. There is thus a widely recognized need for, and it would be highly advantageous to have, compositions and means for preventing or reducing any tobacco-associated damage.

In a search for novel agents for reducing or preventing tobacco-associated damages, the present inventors have surprisingly uncovered that penicillamine is highly effective in ameliorating tobacco-associated damage. As described in detail in the Examples section that follows, treatment with penicillamine substantially increased the survival rate of human lung cancer cells exposed to cigarette smoke, both in the presence and absence of saliva. As further described in the Examples section that follows, a synergistic effect was observed when penicillamine was used in combination with desferal. In addition, penicillamine was found to act as an anti-oxidant, by reducing the production of free radicals, as demonstrated by the prevention of carbonyl production following exposure to CS, both in the absence and presence of saliva. Carbonyls are considered the ultimate markers for protein oxidation and their prevention indicates an action of either antioxidants or anti-aldehyde agents. Penicillamine was also found to protect against CS-induced reduction of the mitochondrial membrane potential, which is a hallmark of apoptosis. Penicillamine was also found to protect against the decrease in the p53 level of expression, inflicted by the synergistic effect of CS and saliva.

While analyzing the results obtained studies conducted, the present inventors have gained some meaningful insights for the mechanisms involved in the carcinogenesis of CS-induced cancers. For a detailed description, see the Examples section that follows. Accordingly, the present inventors have concluded, based on the data obtained, that chelating agents that can remove and/or inhibit the activity of metal ion of redox active metals, act as efficient therapeutic agents for treating or preventing tobacco-induced damages.

Thus, redox active metal chelating agents (e.g., penicillamine), according to the present embodiments, can be efficiently utilized in the manufacture of articles of manufacturing (e.g., tobacco products, filters, tobacco packaging materials and the like), of pharmaceutical compositions, of oral compositions, of medical devices and of medicaments for reducing or preventing tobacco-associated damage.

Herein throughout, the term “agent” encompasses a metal ion chelating agent, as described herein, and penicillamine, including structural analogs thereof, as described herein.

The term “additional agent” is used to describe agents used in addition to the described metal ion chelating agents, penicillamine and structural analogs thereof, as described herein. The additional agent can be, for example, an additional metal ion chelating agent, different from the “agent” described herein.

As used herein, the phrase “metal ion chelating agent”, which is also referred to interchangeably as “metal chelator” or “metal ion chelator”, describes a compound that is capable of forming a stable organometallic complex with metal or metal ion, typically by donating electrons from certain electron-rich atoms present in the compound to the electron-poor metal or metal ion.

As is well known in the art, one or more molecules are considered as transition metal chelators if the formation of a cyclic complex of the molecule(s) with an ion of the transition metal results in a “chelate effect”. The phrase “chelate effect” refers to the enhanced stability of a complexed system containing the chelate, as compared with the stability of a system that is as similar as possible but contains none or fewer rings. The parameters for evaluating the chelate effect of a chelate typically include the enthalpy and entropy changes (ΔH and ΔS), according the following equation:

ΔG⁰=ΔH⁰−TΔS⁰=−RT ln β

where β is the equilibrium constant of the chelate formation and hence represents the chelate effect.

Hence, transition metal chelates refer to complexes that include a metal or a metal ion, and one or more chelator(s) complexed therewith, which are characterized by a large β value.

As used herein, the phrase “redox active metal” or “redox-active metal” describes a metal that can assume two or more oxidation states other than an oxidation state 0, and hence, by its capability of switching from one oxidation state to another, often activates processes (e.g., physiological processes) that generate reactive species, such as, for example, reactive free radicals. Redox active metals are known to be involved in physiological processes associated with oxidative stress.

Exemplary redox active metals include, but are not limited to, copper, which can assume the oxidation states +1 and +2; iron, which can assume the oxidation states +2 and +3; and nickel, which assume the oxidation states +1, +2, +3, and in some cases +4. Other redox active metals are also contemplated.

In some embodiments, the redox active metal is copper.

As used herein, the phrase “copper chelating agent”, which is also referred to interchangeably as “copper chelator”, describes a compound that is capable of forming a stable organometallic complex with copper or copper ion, typically by donating electrons from certain electron-rich atoms present in the compound to the electron-poor copper or copper ion.

Representative examples of copper chelators include polyamine compounds, including linear and cyclic polyamine compounds.

Additional examples of copper chelating agents include, but are not limited to penicillamine, trientine, both being FDA-approved drugs, ethylendiamine, diethylenetriamine, triethylenetetramine, triethylenediamine, aminoethylethanolamine, aminoethylpiperazine, pentaethylenehexamine, triethylenetetramine, captopril, N,N′-bis(3-aminopropyl)-1,3-propanediamine, N,N′-Bis(2-aminoethyl)-1,3-propanediamine, 1,7-dioxa-4,10-diazacyclododecane, 1,4,8,11-tetraazacyclotetradecane-5,7-dione, 1,4,7-triazacyclononane, 1-oxa-4,7,10-triazacyclododecane, 1,4,8,12-tetraazacyclopentadecane and 1,4,7,10-tetraazacyclododecane.

These chelating agents are known also as chelators of redox active metals such as nickel and iron.

Additional examples of suitable chelating agents include, but are not limited to, staurosporine aglycone, 4,5-dianilinophthalimide, 1,10-phenanthroline, and 1,2-diaminobenzene, including derivatives or structural analogs of any of the foregoing chelating agents.

By “derivatives” it is meant, for example, that the compound includes additional substituents or that one or more substituents are replaced by other substituent(s). The substituents can be as described herein for a structural analog of penicillamine. Preferred substituents are such that enhance the chelating effect of the compound, including, but not limited to, alkyl substituents of amino groups, or electron donating groups (e.g., alkoxy, thioalkoxy, and the like) as substituents of an aromatic ring.

Penicillamine, in addition to its copper chelating effect, has also been shown to act as an immunosuppressor, and as an inhibitor of NF-kB and AP-1, thus being recognized as an anti-inflammatory agent.

As demonstrated in the Examples section that follows, penicillamine was found to be highly effective in decreasing the level of cell death upon exposure to cigarette smoke. Penicillamine was highly active both in cells incubated with and without saliva. Penicillamine was shown to act in synergy with desferal, particularly in cells incubated in the presence of saliva.

As discussed hereinabove, chelating agents such as iron chelating agents and cyanide agents were shown to reduce cell death upon exposure to tobacco smoke, in the presence of saliva, thus suggesting that their activity is related to salivary enzymes.

The present findings, which show an effective activity of penicillamine both in the presence and absence of saliva, may suggest that the beneficial effect of penicillamine in reducing or preventing damages caused by exposure to tobacco, results from its immunosuppressant, anti-inflammatory and/or copper chelating activity, as discussed in the Examples section that follows.

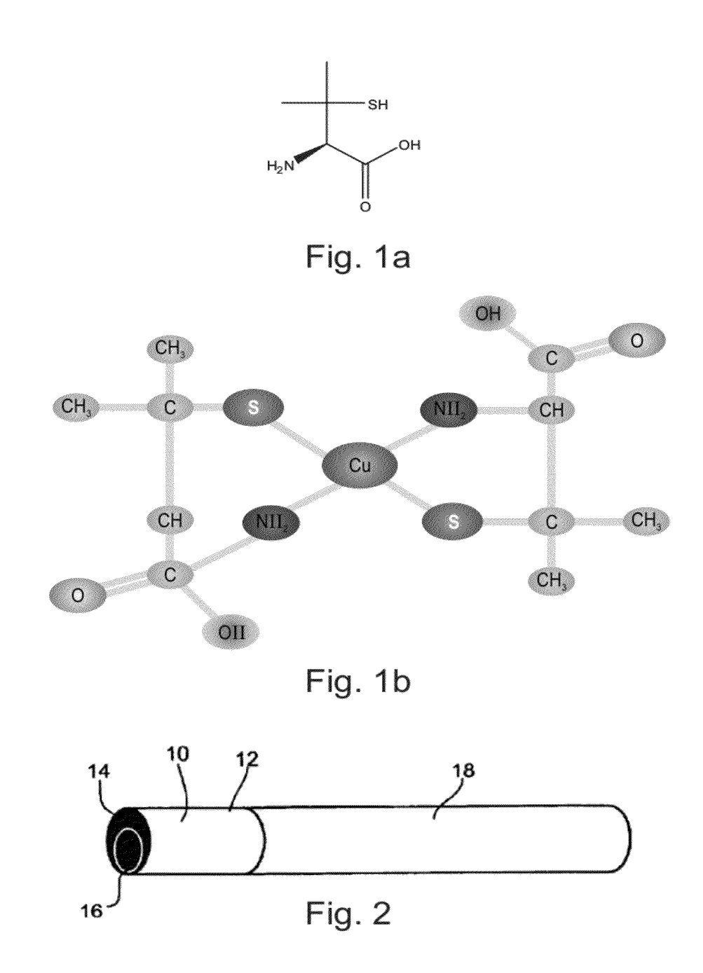

The chemical structure of penicillamine is presented in FIG. 1.

According to some embodiments of the present invention, a penicillamine structural analog is utilized.

As used herein, the phrase “penicillamine structural analog” describes a compound which possesses the main structural features of penicillamine (e.g., an amine group, a thiol group, and a carboxy group, spaced therebetween similarly to penicillamine) and hence may exhibit functional features similar to penicillamine.

According to some embodiments, penicillamine structural analogs are collectively represented by the following general Formula:

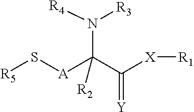

wherein:

X is O or NR₆;

Y is O;

A is CR₇R₈ or CR₇R₈—CR₉R₁₀;

R₁ is selected from the group consisting of hydrogen, a substituted or unsubstituted alkyl, a substituted or unsubstituted cycloalkyl and a substituted or unsubstituted aryl, whereas when anyone of said alkyl, aryl and said cycloalkyl is substituted, the substituent is independently selected from the group consisting of alkyl, cycloalkyl, alkoxy, aryl and aryloxy;

R₂ is hydrogen or alkyl;

R₃ and R₄ are each independently selected from the group consisting of hydrogen, alkyl, alkenyl, cycloalkyl and aryl, or, alternatively, R₃ and R₄ are linked therebetween so as to form a five- or six-membered nitrogen-containing heteroalicyclic ring, or, alternatively, at least one of R₃ and R₄ forms a five- or six-membered heteroalicyclic ring with R₅;

R₅ is selected from the group consisting of hydrogen, a substituted or unsubstituted alkyl, a substituted or unsubstituted cycloalkyl and a substituted or unsubstituted aryl, whereas when anyone of said alkyl, said cycloalkyl and said aryl is substituted, the substituent is independently selected from the group consisting alkyl, cycloalkyl, alkoxy, aryl, aryloxy, carbonyl, aldehyde and carboxy, or, alternatively, R₅ forms a five- or six-membered heteroalicyclic ring with one of R₃ and R₄;

R₆ is selected from the group consisting of hydrogen, a substituted or unsubstituted alkyl, a substituted or unsubstituted cycloalkyl and a substituted or unsubstituted aryl, whereas when anyone of said alkyl, said aryl and said cycloalkyl are substituted, the substituent is independently selected from the group consisting of alkyl and cycloalkyl, or, alternatively, R₆ forms with R₁ a nitrogen-containing five-, six- or seven-membered ring; and

R₇, R₈, R₉ and R₁₀ are each independently selected from the group consisting of hydrogen and alkyl.

It will be appreciated by one of skills in the art that the feasibility of each of the variables (denoted as A, X, Y and R₁-R₁₀) to be located at the indicated positions depends on the valency and chemical compatibility of the substituent, the substituted position and other substituents. Hence, the present embodiments are aimed at encompassing all the feasible substituents for any position.

The term “alkyl”, as used herein, describes a saturated aliphatic hydrocarbon including straight chain and branched chain groups. In some embodiments, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. In some embodiments, the alkyl is a medium size alkyl having 1 to 10 carbon atoms. In some embodiments, unless otherwise indicated, the alkyl is a lower alkyl having 1 to 4 carbon atoms and even 1 to 2 carbon atoms. The alkyl group may be substituted or unsubstituted, as indicated hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated hereinabove.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated hereinabove.

The term “carbonyl” or “carbonate” as used herein, describes a —C(═O)—R′ group, with R′ being hydrogen, alkyl, cycloalkyl or aryl, as defined herein.

The term “aldehyde” describes a carbonyl group in which R′ is hydrogen.

The term “alkoxy” describes both an —O-alkyl and an —O-cycloalkyl group, as defined herein.

The term “aryloxy” describes an —O-aryl, as defined herein.

The term “C-carboxylate” describes a —C(═O)—OR′ group, where R′ is as defined herein.

The term “O-carboxylate” describes a —OC(═O)R′ group, where R′ is as defined herein.

The terms “C-carboxylate” and “O-carboxylate” are referred to herein collectively as “carboxy”.

Each of the alkyl, cycloalkyl and aryl groups in the general formula herein may be substituted by one or more substituents, whereby each substituent group can independently be, for example, alkyl, cycloalkyl, alkoxy, aryl and aryloxy, carbonyl, aldehyde and carboxy, depending on the substituted group and its position in the molecule.

Other substituents, such as heteroaryl, heteroalicyclic, amine, halide, sulfonate, sulfoxide, phosphonate, hydroxy, thiohydroxy, thioalkoxy, thioaryloxy, cyano, nitro, azo, isocyanate, sulfonamide, thiocarbonyl, acyl halide, C-carboxylate, O-carboxylate, N-thiocarbamate, O-thiocarbamate, urea, thiourea, N-carbamate, O-carbamate, C-amide, N-amide, guanyl, guanidine, silyl, and hydrazine, are also encompassed herein, as long as the functionality of the compound remains similar to that of penicillamine.

The term “halide” and “halo” describes fluorine, chlorine, bromine or iodine.

The term “haloalkyl” describes an alkyl group as defined above, further substituted by one or more halide.

The term “S-sulfonamide” describes a —S(═O)₂—NR′R″ group, with R′ as defined herein and R″ being as defined herein for R′.

The term “N-sulfonamide” describes an R′S(═O)₂—NR″— group, where R′ and R″ are as defined herein.

The terms “S-sulfonamide” and “N-sulfonamide” are collectively referred to herein as sulfonamide.

The term “thiocarbonyl” as used herein, describes a —C(═S)—R′ group, with R′ as defined herein.

The term “hydroxyl” describes a —OH group.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an —N═C═O group.

The term “nitro” describes an —NO₂ group.

The term “acyl halide” describes a —(C═O)R″″ group wherein R″″ is halide, as defined hereinabove.

The term “azo” or “diazo” describes an —N═NR′ group, with R′ as defined hereinabove.

The term “C-thiocarboxylate” describes a —C(═S)—OR′ group, where R′ is as defined herein.

The term “O-thiocarboxylate” describes a —OC(═S)R′ group, where R′ is as defined herein.

The term “N-carbamate” describes an R″OC(═O)—NR′— group, with R′ and R″ as defined herein.

The term “O-carbamate” describes an —OC(═O)—NR′R″ group, with R′ and R″ as defined herein.

The term “O-thiocarbamate” describes a —OC(═S)—NR′R″ group, with R′ and R″ as defined herein.

The term “N-thiocarbamate” describes an R″OC(═S)NR′— group, with R′ and R″ as defined herein.

The term “S-dithiocarbamate” describes a —SC(═S)—NR′R″ group, with R′ and R″ as defined herein.

The term “N-dithiocarbamate” describes an R″SC(═S)NR′— group, with R′ and R″ as defined herein.

The term “urea”, which is also referred to as “ureido”, describes a —NR′C(═O)—NR″R′″ group, where R′ and R″ are as defined herein and R′″ is as defined herein for R′ and R″.

The term “thiourea”, which is also referred to as “thioureido”, describes a —NR′—C(═S)—NR″R′″ group, with R′, R″ and R′″ as defined herein.

The term “C-amide” describes a —C(═O)—NR′R″ group, where R′ and R″ are as defined herein.

The term “N-amide” describes a R′C(═O)—NR″— group, where R′ and R″ are as defined herein.

The terms “N-amide” and “C-amide” are collectively referred to herein as amide.

The term “guanyl” describes a R′R″NC(═N)— group, where R′ and R″ are as defined herein.

The term “guanidine” describes a —R′NC(═N)—NR″R′″ group, where R′, R″ and R′″ are as defined herein.

The term “hydrazine” describes a —NR′—NR″R′″ group, with R′, R″, and R′″ as defined herein.

The term “amine” describes a —NR′R″ group, with R′ and R″ as described herein.

The term “silyl” describes a —SiR′R″R′″ group, whereby each of R′, R″ and R′″ are as defined herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

In some embodiments, X is O, such that the compound comprises a carboxy group. When R₁ is hydrogen, the compound comprises a carboxylic acid group. When R₁ is other than hydrogen, the compound comprises an ester.

In some embodiments, X is NR₆, such that the compound comprises an amide group. In cases where R₁ and R₆ form a nitrogen-containing heteroalicyclic group, the amide is a cyclic amide.

The ester or amide can be further substituted, as described herein.

In some embodiments, R₃ and R₄ are both hydrogen, thus forming a primary amine at the indicated position.

In cases where one of R₃ and R₄ is other than hydrogen, the compound comprises a secondary amine at the indicated position. In cases where both R₃ and R₄ are other than hydrogen, a tertiary amine is present. In cases where R₃ and R₄ are linked therebetween to form a heteroalicyclic ring, a cyclic amine is present. The amine can comprise an additional group, represented by the indicated substituents.

In cases where R₅ is hydrogen, the compound comprises a thiol moiety. In cases where R₅ is alkyl or cycloalkyl, the compound comprises a thioalkoxy moiety. In cases where R₅ is aryl, the compound comprises a thioaryloxy moiety. Each of the thioalkoxy or thioaryloxy can further comprise an additional group, represented by the indicated substituents on the alkyl, aryl and cycloalkyl groups that form a part of thioalkoxy and thioaryloxy.

The variable A in the general formula above represents a spacer between the amine moiety and the thiol, thioalkoxy or thioaryloxy moiety. The spacer includes 1 or 2 carbon atoms, being optionally substituted as indicated hereinabove.

The configuration of the carbon atom that bears the R₂ group, or of any other chiral carbon atom that may be present in the molecule can be R configuration or S configuration. In one embodiment, the carbon atom that bears the R₂ group has R configuration.

The agent described herein (e.g., a penicillamine structural analog or any of the metal ion chelating agents described herein) is preferably selected as being capable of suppressing an innate immune activity in a subject using the article of manufacturing.

The agent (e.g., a penicillamine structural analog or any of the metal ion chelating agents described herein) is further preferably selected as being capable of inhibiting inflammation in a subject using the article of manufacturing.

The present embodiments further encompass pharmaceutically acceptable salts of the agents described herein.

The phrase “pharmaceutically acceptable salt” describes a charged species of the parent compound and its counter ion, which is typically used to modify the solubility characteristics of the parent compound and/or to reduce any significant irritation to an organism by the parent compound, while not abrogating the biological activity and properties of the administered compound. Examples, without limitation, include an acid additional salt of an amine group.

The present invention further encompasses prodrugs, solvates and hydrates of the agents described herein.

As used herein, the term “prodrug” refers to a molecule, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo. An example, without limitation, of a prodrug would be a compound, having one or more carboxylic acid moieties, which is administered as an ester (the “prodrug”). Such a prodrug is hydrolysed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the agent described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

According to one aspect of embodiments of the present invention there is provided an article of manufacturing which comprises tobacco and a tobacco packaging material, wherein at least a portion of the tobacco and/or the tobacco packaging material comprises an agent as described herein.

According to another aspect of embodiments of the present invention there is provided an article of manufacturing which comprises tobacco and an agent as described herein being incorporated in at least a portion of the tobacco.

According to another aspect of embodiments of the present invention there is provided an article of manufacturing which comprises a tobacco packaging material and an agent as described herein being incorporated in at least a portion of the tobacco packaging material.

The term “agent” refers to any of the metal ion chelating agents (e.g., copper chelating agents), penicillamine and structural analogs thereof described herein.

In one embodiment relating to the articles of manufacturing described herein, that portion of tobacco and/or the tobacco packaging material which comprises the agent is in contact with an aerodigestive tract of a subject using the article of manufacturing.

As used herein the term “tobacco” refers to any tobacco species (e.g., crude or extract) which is compatible with human use. The agent can be incorporated in the tobacco (or a portion thereof), by mixing, dipping, spraying, coating, or any other chemical or physical attachment.

On top of tobacco, the present embodiments also envisage the use of the agents described herein (in line with the above described aspects) with other smoked, dipped, chewed, snuff or snused herbs, compatible with human consumption and which cause a damage similar to that damage induced by tobacco, as detailed hereinunder.

As used herein, the phrase “tobacco packaging material” refers to any auxiliary means which packages the tobacco or facilitates its consumption (carrier). Examples include, but are not limited to, rolling paper, snus bags, filter paper, tin sheets and the like.

Thus, for example, the agent may be impregnated in (attached to, absorbed in, coated with) a filter paper which comes in direct contact with the aerodigestive tract.

The articles of manufacturing described herein can therefore be, for example, tobacco products such as smoking products (e.g., cigarettes, non-filter cigarettes, cigars, and other tobacco products as described hereinabove) or products used in the manufacturing of tobacco products (e.g., cigarette filters, rolling papers and the like).

FIG. 2 illustrates a cigarette filter configuration which is referred to hereinunder as a cigarette filter 10. Cigarette filter 10 is constructed of a paper lining 12 and a filter core 14 which is composed of glass fiber and is positioned adjacent to a tobacco filling 18. To enable effective delivery the agent of the present invention can be disposed as an aqueous emulsion within a rupturable capsule 16 positioned at the front of filter core 14. Alternatively, the agent may also be dispersed, impregnated in tobacco filling 18 or provided throughout in droplets or beadlets through the employment of gelatin or other colloidal materials, so that the agent can be easily entrained by the smoke passing through filter core 14. Such filters have been previously described, for example, in U.S. Pat. Nos. 3,667,478 and 3,339,558, the teachings of which are herein incorporated by reference as if fully set forth herein.

Alternatively, the rolling paper may be treated with the agent such that the agent is confined to that region of the paper which comes in contact with the aerodigestive tract (say about 1 cm margines).

Such tobacco filters can be used as follows: prior to lighting up, pressure is applied to rupturable capsule 16, so that the released agents are dispersed within filter core 14, whereby the agent is accessible to the cigarette smoke passing through.

Thus, in some embodiments of the present invention, the articles of manufacturing described herein are preferably designed and configured so as to enable physico-chemical interaction between the agent and the tobacco smoke. In some embodiments, the articles of manufacturing are designed and configured so as to enable release of the agent therefrom when in use by a subject.

As used herein, “aerodigestive tract” refers to saliva-lined tissues such as the lips, mouth, buccal cavity, tongue, oropharynx, throat, larynx, esophagus, upper digestive tract, saliva glands, saliva, as well as the similar mucous-lined tissues of the respiratory tract, such as the respiratory mucosa, alveoli, trachea, and lungs.

Further according to the present embodiments, there is provided an article of manufacturing, being a filter, which comprises an agent as described herein and which is designed and configured so as to enable release of the agent therefrom when in use by a subject.

In some embodiments, the filter is designed and configured as a tobacco smoke filter (see, for example, FIG. 2). Such a filter can be incorporated into “filter-tip cigarettes”, cigarette holders, gas-masks, protective face-masks, and air-conditioning unit filters.

In some embodiments, any of the articles of manufacturing described herein further comprises an additional agent that is capable of reducing or preventing a tobacco-associated damage, as described herein, in a subject using the article of manufacturing.

The additional agent can be incorporated (or impregnated) in the tobacco or the tobacco packaging material or in the above-described filter.

Since tobacco-associated damage often involves oxidative damage, exemplary such additional agents are antioxidants. In one embodiment, the antioxidant is glutathione (GSH). As demonstrated in the Examples section that follows, GSH was found to be highly active in reducing cell death induced by exposure to tobacco smoke.

The additional agent can further be a cyanide (CN⁻) chelator, which can be used to treat tobacco-associated loss of OPO activity. An example of such a chelator is OH—CO, also known as the non-cyanide-bound form of cyanocobalamin, hydroxocobalamin or vitamin B12a. Other examples include, but are not limited to, epselen, vitamins A, C and E, selenium compounds, flavenoids, quinones (e.g., Q10, Q9), retinoids and carotenoids.

Preferably, the CN⁻ chelator (e.g., OH—CO) is administered in a manner which enables establishment of a concentration of 0.5-2 mM, preferably 1 mM in body fluids, such as saliva.

Cyanide chelators can be effectively employed to prevent or reduce tobacco-associated damage in the aerodigestive tract since they act to sequester cyanide which is injurious to OPO.

Other antioxidants include redox-active metal ion chelators, e.g., redox-active iron chelators (also referred to herein as iron chelating agents). Examples include deferoxamine, and zinc-desferioxamine.

The chelating agent deferoxamine is also known as DES, desferal and desferioxamine.

Redox-active metal ion chelators are used in a manner which enables establishment of about 1 mM concentration in body fluids (e.g., saliva). Preferably, deferoxamine is administered in a manner which enables establishment of a concentration of about 1 mM, more preferably about 5 mM in body fluids. More preferably, a mixture of deferoxamine and GSH is used in a ratio of about 1:1, preferably 5:1, respectively. When used in combination, deferoxamine and GSH body fluid concentrations of about 1 mM each are desirable although a deferoxamine concentration of 5 mM and a GSH concentration of 1 mM are also therapeutically effective.

In one embodiment, the additional active agent is desferal.

Preferably, desferal is used in combination with penicillamine, to thereby exert a synergistic effect.

The articles of manufacturing described herein can further comprise at least one flavorant such as, but not limited to, wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, and cinnamon leaf oil.

Any of the agents and additional agents described herein may be introduced to the article of manufacturing as described above (e.g., snuff), such as in the form of a dry powder, either as a mixture of antioxidants, or as a complex in protective liposomes, nanospheres or other acceptable delivery vehicles. This powder may be added in the final process of manufacturing and may also contain suitable flavors or fragrances as not infrequently used in this industry.

As discussed hereinabove and is exemplified in the Examples section that follows, the agents described herein (metal ion chelating agents, copper chelating agents, penicillamine and structural analogs thereof) are highly efficient in reducing or preventing damages caused by tobacco (e.g., by cigarette smoke), both cellular damage (e.g., cell death) and macromolecular damage (e.g., protein carbonylation), and exhibit their activity also in the absence of saliva.

Thus, according to another aspect of embodiments of the present invention there is provided a method of treating or preventing tobacco-associated damage in a subject in need thereof, which is effected by administering to the subject a therapeutically effective amount of a metal ion chelating agent, as described herein. In some embodiments, the chelating agent is a copper chelating agent. In some embodiments, it is penicillamine or a structural analog thereof, as described herein.

In some embodiments, administering the agent as described herein is effected via, for example oral, rectal, transmucosal, transdermal, topical, intestinal or parenteral delivery, including intramuscular, subcutaneous and intramedullary injections as well as intrathecal, direct intraventricular, intravenous, intraperitoneal, intranasal, or intraocular injections.

Subject treatable by the method described herein include tobacco consumers, as well as secondary tobacco consumers (non-smokers that are exposed to tobacco smoke).

Accordingly, according to another aspect of embodiments of the present invention, there is provided use of any of the agents described herein in the manufacture of a medicament for treating a tobacco-associated damage.

As used herein, the phrase “tobacco-associated damage” described cellular or macromolecular damage which is induced or exacerbated by exposure to tobacco consumption. The phrase “tobacco consumption” includes, for example, tobacco smoking (including primary and secondary smoking), chewing, sniffing, and the like, as described hereinabove.

As further discussed hereinabove, the tobacco-associated damage can be a cellular damage, resulting in, for example, cell death, cell malfunction, cell mutation, and the like; or a macromolecular damage, resulting in modification of macromolecules such as lipids, DNA and proteins.

As further discussed hereinabove, tobacco-associated damage typically involves ROS and therefore often involves oxidative damage of cells and cell components. Tobacco consumption often results in protein carbonylation in the plasma.

Being in direct contact with the aerodigestive tract, tobacco consumption results in tobacco-associated damage to mucosal tissues, particularly saliva-lined tissues such as the lips, mouth, buccal cavity, tongue, oropharynx, throat, larynx, esophagus, upper digestive tract, saliva glands, saliva, as well as the similar mucous-lined tissues of the respiratory tract, such as the respiratory mucosa, alveoli, trachea, and lungs.

Tobacco-associated damage, however, can further affect non-mucosal tissues.

As further discussed hereinabove, tobacco-associated damage is typically manifested as various diseases and disorders, including, but not limited to, cardiovascular diseases, chronic obstructive pulmonary disease, lung cancer, as well as other forms of cancer (e.g., aerodigestive tract cancers) and peripheral vascular disease.

Exemplary cardiovascular diseases that are therefore treatable by the agents described herein include, but are not limited to, atherosclerosis; coronary thrombosis, which may lead to a heart attack; cerebral thrombosis, which may lead to collapse, stroke and paralysis; affected kidney arteries, which result in high blood pressure or kidney failure; and blockage of the vascular supply to the legs, which may lead to gangrene and amputation.

Exemplary cancers that are treatable by the agents described herein, in addition to lung cancer, include, but are not limited to, mouth, pharynx, and esophagus cancer, and oral squamous cell carcinoma. Other types of cancers include bladder cancer, cancer of the kidneys, cancer of the pancreas and cervical cancer.

Exemplary chronic obstructive pulmonary diseases (COPD) that are treatable by the agents described herein include, but are not limited to, emphysema and chronic bronchitis. Severe asthma can be deteriorated as a result of exposure to tobacco smoke, and moreover, such am exposure often contradicts the effect of asthma medications.

Other damages associated with tobacco consumption, which are treatable by the agents described herein include, for example, hypertension, fertility problems, retinoic disorders such as macular degeneration and cataracts, ulcers, periodontal diseases, impotence, Diabetes type 2, Back pain, skin ailments such as premature ageing and wrinkling, osteoporosis, earlier menopause, and damaged and/or weakened immune system, as well as leukoplakia, halitosis, acute necrotizing ulcerative gingivitis (“trench mouth”) and oral submucous fibrosis.

In any of the methods and uses described herein, the agent can be utilized in combination with an additional agent. The additional agent can be, for example, an antioxidant as described herein, an agent capable of reducing or preventing a tobacco-associated damage and/or an agent suitable for use in the treatment of a disease or disorder as described herein.

In one embodiment, the additional agent is desferal.

In any of the methods and uses described herein, the agent can be utilized either per se or being formulated into a pharmaceutical composition which further comprises a pharmaceutically acceptable carrier.

Hence, according to still another aspect of the present invention, there are provided pharmaceutical compositions, which comprise one or more of the agents described above and a pharmaceutically acceptable carrier.

As used herein a “pharmaceutical composition” refers to a preparation of one or more of the agents described herein, with other chemical components such as pharmaceutically acceptable and suitable carriers and excipients. The purpose of a pharmaceutical composition is to facilitate administration of a compound to an organism.

Hereinafter, the phrase “pharmaceutically acceptable carrier” describes a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the agents described herein into preparations which, can be used pharmaceutically. Proper formulation is dependent upon the route of administration chosen.

According to some embodiments, the pharmaceutical composition is formulated as a solution, suspension, emulsion or gel.

According to some embodiments, the pharmaceutical composition further includes a formulating agent selected from the group consisting of a suspending agent, a stabilizing agent and a dispersing agent.

For injection, the agents described herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

For oral administration, the agents described herein can be formulated readily by combining the agents with pharmaceutically acceptable carriers well known in the art. Such carriers enable the agents described herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active agent doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the agent(s) may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents described herein are conveniently delivered in the form of an aerosol spray presentation (which typically includes powdered, liquified and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the agents and a suitable powder base such as, but not limited to, lactose or starch.

The agents described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the agents described herein in water-soluble form. Additionally, suspensions of the agents may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the agents to allow for the preparation of highly concentrated solutions.

Alternatively, the agents may be in powder form for constitution with a suitable vehicle, e.g., sterile, pyrogen-free water, before use.

The agents described herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of an agent as described herein effective to prevent, alleviate or ameliorate symptoms of tobacco-associated damage or prolong the survival of the subject being treated.

Determination of a therapeutically effective amount is well within the capability of those skilled in the art, especially in light of the detailed disclosure provided herein.

For any agent utilized in the methods and uses of the invention, the therapeutically effective amount or dose can be estimated initially from activity assays in animals. For example, a dose can be formulated in animal models to achieve a circulating concentration range that includes the IC₅₀ as determined by activity assays (e.g., the concentration of the test agent, which achieves a half-maximal reduction in cell death upon exposure to cigarette smoke). Such information can be used to more accurately determine useful doses in humans.

Toxicity and therapeutic efficacy of the agents described herein can be determined by standard pharmaceutical procedures in experimental animals, e.g., by determining the EC₅₀, the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present embodiments may, if desired, be presented in a pack or dispenser device, such as an FDA (the U.S. Food and Drug Administration) approved kit, which may contain one or more unit dosage forms containing the active agent. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising an agent as described herein, formulated in a compatible pharmaceutical carrier may also be prepared, placed in an appropriate container, and labeled for treatment of an indicated condition, as is detailed hereinabove.

Thus, according to an embodiment of the present invention, the pharmaceutical composition packaged in a packaging material and identified in print, in or on the packaging material, for use in the treatment of a tobacco-associated damage, as described herein.

In some embodiments, the pharmaceutical composition further comprises an additional agent. The additional agent can be, for example, an antioxidant as described herein, an agent capable of reducing or preventing a tobacco-associated damage and/or an agent suitable for use in the treatment of the indicated disease or disorder as described herein. An exemplary antioxidant is desferal.

According to another aspect of the present invention there is provided an oral composition which comprises an agent as described herein. The oral composition can be in the form of a toothpaste, powder, liquid dentifrice, mouthwash, denture cleanser, chewing gum, lozenge, paste, gel or candy and can further comprise at least one flavorant such as wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, and cinnamon leaf oil.

Exemplary chewing gum compositions are described in U.S. Pat. No. 5,922,346, which is incorporated by reference as if fully set forth herein.

The chewing gums, gels or pastes of these embodiments may include bicarbonates with thickening agents in a concentration from 0.5% to 5.0% by weight. Exemplary thickeners with bicarbonate and zinc salts include, but are not limited to, chicle, xanthan, arabic, karaya or tragacanth gums. Alginates, carrageenans and cellulose derivatives such as sodium carboxymethyl, methyl, or hydroxy ethyl compounds are appropriate for the intended preparations; surfactants and abrasives may also be included. Alcohols will otherwise be avoided for their known risk factor for oral cancers. In order to decrease dental cavities and add flavor, without using metabolizable sugars, sweetening agents as saccharin, sodium cyclamate, sorbitol, aspartamane and others may be used in concentrations from 0.005% to 5.0% by weight of the total composition. Xylitol has been shown to prevent dental caries and decrease gum disease, in part by reducing the putative oral bacteria, especially Streptococcus mutants.

Gels and dentifrices may contain fluoride anticaries compounds. These fluoride compounds, such as salts of sodium, potassium, calcium, magnesium, stannous and others have been known to protect teeth from developing cavities. Fluorides may be present in various amounts in the gels, pastes, gums or lozenges ranging from 0.01% to 3.0% by weight, preferably from 0.05% to 2.0% by weight, most preferably from 0.1% to 1.2% by weight. These sources of stabilized fluoride are taught in U.S. Pat. No. 5,372,802.

The agents described herein can also be incorporated into additional articles. These include, for example, various medical devices for delivering the agent to or applying the agent on a desired bodily site.

As used herein, the phrase “bodily site” includes any organ, tissue, membrane, cavity, blood vessel, tract, biological surface or muscle, which delivering thereto or applying thereon the agents described herein is beneficial.

Exemplary medical devices are those configured to deliver the agent by topical application, (e.g., an adhesive strip, a bandage, an adhesive plaster, and a skin patch).

The agents can be incorporated in the device structure by any methodology known in the art, depending on the selected nature of the device structure. For example, the agents can be entrapped within a porous matrix, swelled or soaked within a matrix, or being adhered to a matrix.

In any of the articles, compositions and devices described herein, the agent can be utilized in combination with an additional active agent, as described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Experimental Methods

Materials:

Tobacco smoke was obtained from popular commercial cigarettes containing 14 mg of tar and 0.9 mg of nicotine (‘Time’ cigarettes, Dubek Ltd., Tel Aviv, Israel), and was generated as described hereinbelow.

Human non-small cell lung cancer NCI-H1299 cells were used as described by the American Type Culture Collection.

Culture medium included DMEM with L-glutamine, supplemented with fetal bovine serum (10%), penicillin-streptomycin solution (1%).

The cells were grown at 37° C. in 5% CO₂.

Culture medium ingredients were supplied by Beit HaEmek-Biological Industries, Israel.

Hydroxocobalamin acetate, D(−)Penicillamine, Defrox amine Mesylate (Desferal-DES), thiol-glutathione (GSH) and MTT (Thiazolyl Blue Tetrazolium Bromide) salt were purchased from Sigma-Aldrich, Israel.

Cell Death Kit (Cell Death Detection ELISA^{PLUS-TM} KIT) was purchased from Roche diagnostics, Germany.

[3H]PK 11195 Clonazepam and diazepam were used as ligands.

BioCell Protein Carbonyl Assay Kit was purchased from BioCell Corporation, New Zealand.

Protein levels were measured by the Bradford method (1976) [Bradford M. M. 1976. Anal. Biochem. 72:248-254] using BSA as a standard.

Saliva Collection:

Whole saliva was collected from healthy volunteers (20-60 years) under non-stimulatory condition, at least 1 hour after last eating, between 8:00-12:00 AM. The volunteers were asked to generate saliva in their mouths and to spit it into a wide test tube for no more then 20 minute. The obtained whole saliva was centrifuged at 1200×g for 15 minutes to remove cell debris and palate cells, and the supernatant was used for further applications.

Study Design:

Experiments were conducted using H1299 human lung cancer cells.

Four control groups, not exposed to tobacco smoke (referred to herein as TS or alternatively, as CS, standing for “cigarette smoke”) were compared to four study groups (similar groups which were also exposed to CS), as detailed hereinafter. The four control groups included the following: a group where the cells were incubated in medium alone (M), a group where the cells were incubated in medium supplemented with whole saliva (M+saliva), a group where the cells where incubated in medium and D-penicillamine (PenA M) and a group where the cells were incubated in medium supplemented with saliva and D-penicillamine (PenA M+saliva). Accordingly, the four study groups were exposed to the same conditions and further exposed to CS (as detailed hereinafter), and are denoted CS, CS+saliva, PenA CS and PenA CS+saliva. D-penicillamine was used at a concentration of 1 mM or 5 mM.

In other sets of experiments, H1299 cells were similarly treated, using, instead of (or in addition to) PenA, GSH, or DES, at the indicated concentration.

Exposure to Cigarette Smoke (CS) with/without Saliva:

During smoke exposure, the cells were put into a smoking sealed chamber (33.5 cm³) with CS pressure of 0.3 Bar. Control cells were subjected to the same procedures, but exposed only to fresh air. Cells were exposed to CS for one “puffs” every 15 minute for a 2 hours (120 minutes) period (unless indicated otherwise). Cells were used when it becomes confluent and the medium (with/without saliva) volume was compatibly to the dish in use.

Whole Cell Extract Preparation:

Cells medium, after CS exposure, was moved and the cultured cells were collected by scraping. Samples were centrifuged at 1200×g for 10 minutes. The cell pellets were lysed with 50-150 μL lysis buffer containing 1% Triton X-100, 1 tablet/10 ml protease inhibitor and 0.1% SDS dissolved in PBS, pH 7.4, for 30 minutes on ice. Cell extracts were thereafter centrifuged at 12,000×g for 10 minutes at 4° C. The supernatants were transferred into 1.5 ml eppendorf tube and stored at −20° C. until used. Protein concentration was measured using the Bradford method, as described herein.

H1299 Cells Survival:

The H1299 cells viability was measured at various time points by Trypan Blue exclusion test, both in exposed and control cells.

The medium covering the dish was collected and cells were trypsinized and centrifuged at 1200×g for 10 minutes. Cell pellets were re-suspended in 1 mL of medium and a sample was collect for cell counting. Cells were stained with the vital dye Trypan Blue at final concentration of 0.25% and were placed on hemocytometer. Visual counting was preformed by inverted microscope.

Protein Carbonyl Assay:

Protein carbonyl concentration was determined by enzyme-linked immunosorbent assay (ELISA), using the Zentech PC Test Kit (Zenith Technology, Dunedin, New Zealand). Briefly, protein cell extractions were allowed to react with a dinitrophenylhydrazine (DNP) solution (200 μl). The DNP-reacted proteins bound non-specifically to an ELISA plate, and the unconjugated DNP and non-protein entities were washed away. The adsorbed DNP-protein was then probed with an anti-DNP-biotin antibody, followed by a streptavidin-linked HRP probe. Then the chromatin reagent that contained peroxide was added to catalyze the oxidation of TMB. Finally, the reaction was stopped by the addition of a stopping reagent (acid, provided with the kit), and the absorbance was measured for each well at 450 nm using a spectrophotometer. Along with controls and samples, protein carbonyl standards were also included in the assay. The content of the carbonyl protein in the mitochondrial samples was determined as pmol/mg protein, using the standard curve.

Flow Cytometric Analysis (FACS):

FACS was used to monitor cell-cycle and cell-viability after CS exposure with/without saliva presence. After cells were exposed to ROS attack, medium covering the cells was removed and cells were trypsinized. For cell viability, cell suspension was centrifuged at 1200×g for 10 minutes and the pellets were re-suspended in 0.5 ml of 10 μg/ml Propidium iodide (PI) and incubated for 15 minutes at dark condition, 4° C. The cell suspensions were thereafter analyzed with a flow cytometer, using CellQuest software.

For mitochondrial oxidative damage, pellets were re-suspended in 0.5 ml of 10 μg/ml 10-N-nonyl acridine orange (NAO) and incubated for 30 minutes at 37° C. and dark condition. Mitocondrial membrane potential was measured after staining cells with 1 μg/ml JC-1 (5,5′,6,6′-tetrachloro-1,1′,3,3′-tetraethylbenzimidazolyl carbocyanine iodide; Molecular Probes) for 30 minutes at 37° C. in the dark. Uncoupling with carbonyl cyanide p-trifluoromethoxyphenylhydrazone (FCCP) (20 lM for 30 minutes) was used as positive control.

Western Blot Analysis:

Western blot analysis was used to assay the protein expression levels of the P₅₃, using β-actin signal as a loading reference. Samples with equal amount of protein (20 μg protein/lane) were prepared in 2× sample buffer (0.125 M 2-amino-2-(hydroxymethyl)propane-1,3-diol (Tris)-HCl, pH 6.8, glycerol (20% v/v), SDS (4% w/v), 0.14 M β-mercaptoethanol and bromophenol blue (0.005% w/v)). The samples were boiled for 10 minutes and subjected to electrophoresis through 12% SDS-polyacrylamide gel in running buffer, or stored at −20° C. until further use. The gels were then electrophoretically transferred to nitrocellulose membranes for 90 minutes at 90 W, in transfer buffer containing 20 mM Tris-HCl, pH 6.8, 150 mM Glycine and 20% (v/v) methanol. Blots were blocked in 5% non-fat milk in PBS-T (62.5 mM sodium phosphate buffer, pH 7.4, containing 100 mM NaCl and 0.1% (v/v) Tween-20) and then incubated for 2 hours or overnight at 4° C. with rabbit antihuman p53 1:5000 or mouse antihuman β-actin 1:15000. After three washes in PBS-T, the membranes were incubated with IgG secondary antibody linked to horseradish peroxidase, anti-rabbit IgG 1:3000 or antimouse IgG 1:5000 in PBS-T for 1 hour at room temperature. Binding of antibodies to the membrane was detected with an EZ-ECL-detection reagent, X-Omat blue XB-1 Kodak scientific Imaging Film for 10 seconds-15 minutes.

Cell Proliferation Assay:

Cells were seeded in a 24-well plate. 500 μL of MTT reagent solution in cells medium were added into wells being assayed, to final concentration of 0.5 mg/ml. Cells were incubated at 37° C. for 1 hour. At the end of the incubation period, the medium was removed. Dye was solubilized with 0.5 ml DMSO (Sigma, Israel) for 30 minutes at room temperature. Dye solution with the cells was transferred into 1.5 ml eppendorf tube and centrifuged at 1200×g for 5 minutes. 100 μL of the supernatant dye was transferred to 96-well plate. Absorbance of the converted dye was measured at a wavelength of 570 nm with background subtraction at 650 nm.

Apoptosis:

To determine the effect of ROS attack on apoptosis, Cell Death Kit was used. Cells were scraped from the culture dishes, and centrifuged at 1200 g for 10 minutes. Cell pellets were re-suspended with lysis buffer according to the manufacturer's instructions. The lysate was centrifuged at 200 g for 10 minutes. A fraction of the supernatant was transferred to streptavidin-coated microtiter plate modules. Immunoreagent was added (antihistone-biotin and anti-DNA-peroxidase in incubation buffer), and after incubation with gentle shaking for 2 hours, the modules were rinsed three times with incubation buffer. Then, the signal for apoptosis was measured following incubation for 15 minutes with 2,2-azino-bis-[3-ethylbenzothiazoline]-6-sulfonic acid (ABTS) solution, according to the manufacturer's instructions. The level of staining by the ABTS substrate was determined with an ELISA reader, at the wavelength of 405 nm. Reference absorbance was measured using a 490 nm wavelength. The ABTS solution by itself was used as a negative control, positive control was supply by the manufacturer.

Lipid Peroxidation Assay (TBARS):

Lipid peroxidation was quantified by determining 2-thiobarbituric acid reactive substance (TBARS) formation according to the method described by Buege and Aust [Methods in Enzymol. 52, 302-310, 1978] with some modification. H1299 cells (2×10⁴ cells) were homogenized 1 ml of PBS. 1 ml of 2-thiobarbituric acid (TBA) reagent consisting of 0.375% TBA, 15% trichloroacetic acid, and 0.25 N HCl was added to 0.5 ml of cell homogenat. The mixture of cell suspension and TBA reagent was heated at 100° C. for 20 minutes, chilled quickly in ice-water to room temperature, and centrifuged at 1,500 g for 10 minutes. The supernatant was measured at 535 nm with a spectrophotometer.

[³H]PK 11195 Binding Assays:

[³H]PK 11195, an isoquinoline carboxamide derivative, was used for binding studies. PK 11195 is a specific PBR ligand. Cells were scraped from the culture dishes, washed with phosphate-buffered saline (PBS), and centrifuged at 1200 g for 10 minutes. Then the cell pellets were re-suspended in 1 mL of 50 mM phosphate buffer, pH 7.4, and centrifuged at 1200 g for 10 minutes. Binding assays contained 400 μL of cell membrane (0.4 mg of protein/mL) in the absence (total binding) or presence (nonspecific binding) of 1 μM unlabeled PK 11195, up to a final volume of 500 μL. After incubation for 80 minutes at room temperature, samples were filtered under vacuum over Whatman GF/B filters and washed three times with phosphate buffer. Filters were placed in vials containing 4 mL of Opti-Fluor (Packard, Groningen, The Netherlands) and counted for radioactivity in a scintillation counter after 12 hours. The maximal number of binding sites (Bmax) and equilibrium dissociation constants were calculated from the saturation curve of [³H] PK 11195 binding, using Scatchard analysis.

Statistical Analysis:

Results for statistical analysis were obtained from the control subgroup (H1299 cells in medium) and from the various treatment subgroups (with/without saliva and/or exposure to CS and/or additions of 1 mM or 5 mM exogenous D-penicillamine).

Means, SDs and SEMs were computed and results between the subgroups were analyzed and compared via one-way analysis-of-variance [Scheffe, H. The Analysis of Variance. New York: John Wiley & Sons. 1959] using the Bonferroni Multiple-Comparison Test Model [Hockberg and Tamhane, Multiple Comparison Procedures. New York: John Wiley & Sons. 1987] to determine significant differences between computed means. The means between each pair of means was analyzed via T-test For Paired Differences and means between each two subgroups were compared via Two Sample T-test For Differences in Means [Gosset Biometrika 1908; 6:1-25].

Results are expressed as the mean+standard error {SD/\sqrt{N}}. Experimental and control groups were usually at n≧5, unless otherwise indicated.

### Experimental Results

Exposure of H1299 Cells to CS and/or Saliva:

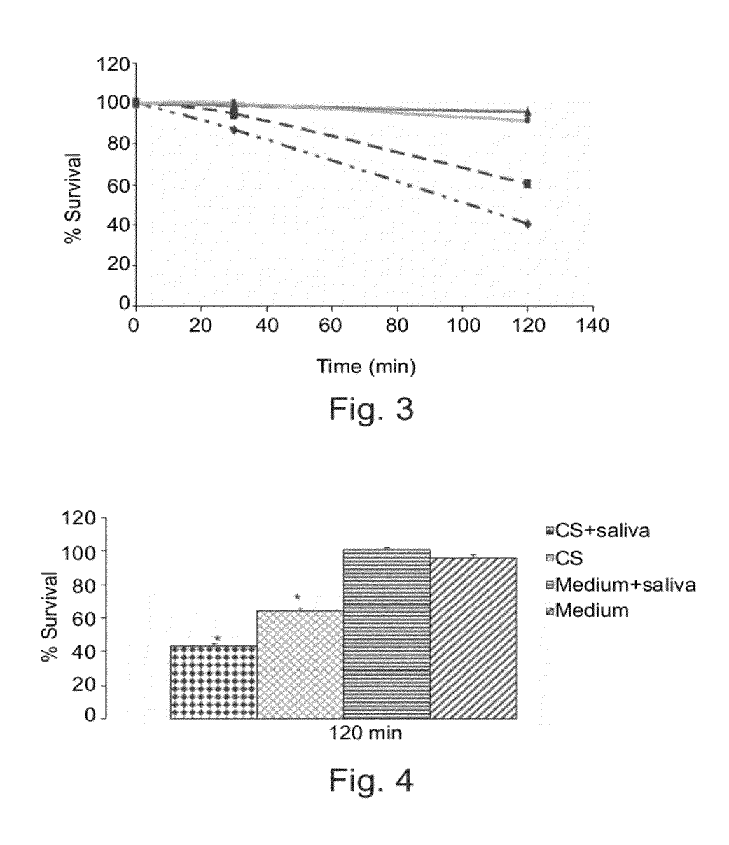

FIGS. 3 and 4 present the effect of saliva and tobacco smoke (=cigarette smoke, CS), either alone or in combination, on the survival rate of H1299 cells, as a function of time (FIG. 3) and at 120 minutes (FIG. 4), and clearly show the synergistic effect of CS and saliva on cells survival.

Modification of CS and/or Saliva Effects by D-Penicillamine (PenA):

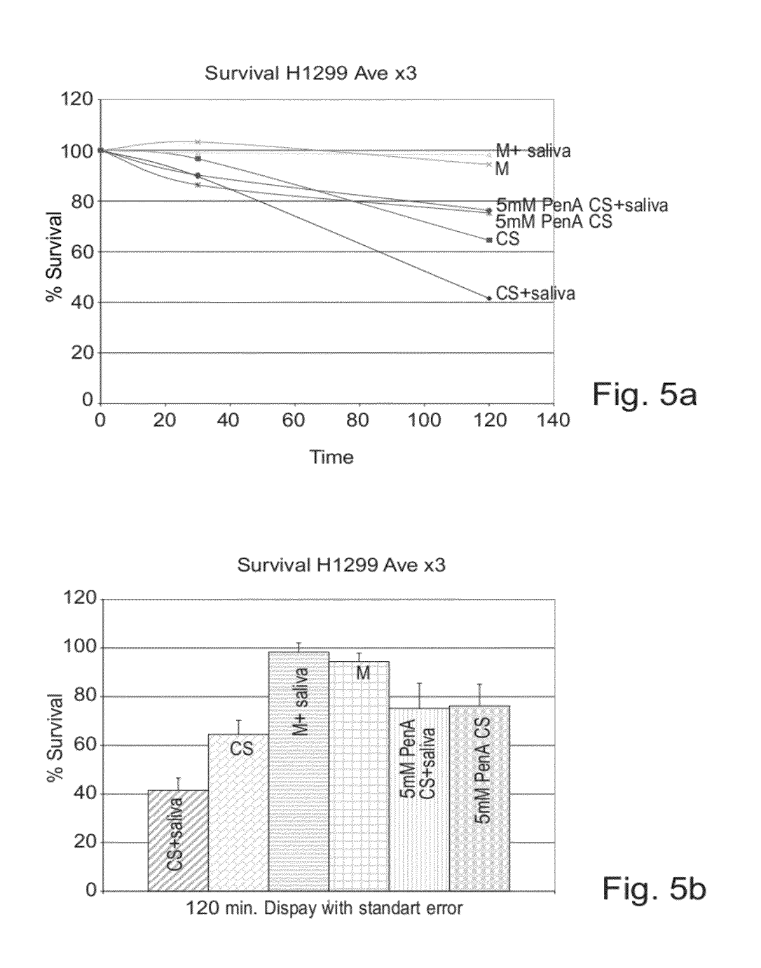

FIGS. 5A and 5B present the effect of 5 mM penicillamine (PenA) on the survival rate of H1299 cells incubated in medium containing PenA or in medium containing 30% (v/v) saliva and PenA, and exposed to CS.

H1299 cells were incubated for 120 minutes with or without the presence of saliva in the culture medium and exposed to CS. Cells were also incubated in the presence of PenA in either low (1 mM) or high (5 mM) concentration prior the exposure of CS. Cell viability was evaluated by Trypan blue exclusion dye assay.

As shown in FIGS. 5A and 5B, during the 120-minutes exposure to CS of the cells in medium alone or in medium supplemented with 30% (v/v) saliva, with or without prior addition of PenA to the medium, no significant survival loss was noted. However, 120 minutes exposure of the cells (in medium) to CS resulted in a time-dependent reduced survival which was most significant after 120 minutes (survival loss of 35.5%, P<0.01). The addition of saliva to the medium while exposing the cells to CS resulted in a lethal synergistic effect as demonstrated by a 58.5% (P<0.01) survival loss.

As shown in FIGS. 5A and 5B, the addition of 5 mM D-penicillamine to cells incubated with or without exposure to CS (PenA+CS and PenA M groups, respectively) partially affected the survival of H1299 cells, such that the cells loss at 120 minutes of exposure to CS in the presence of the PenA was reduced by 25%. Reduction of cells loss was also observed when saliva was supplemented to the medium prior to the exposure to CS.

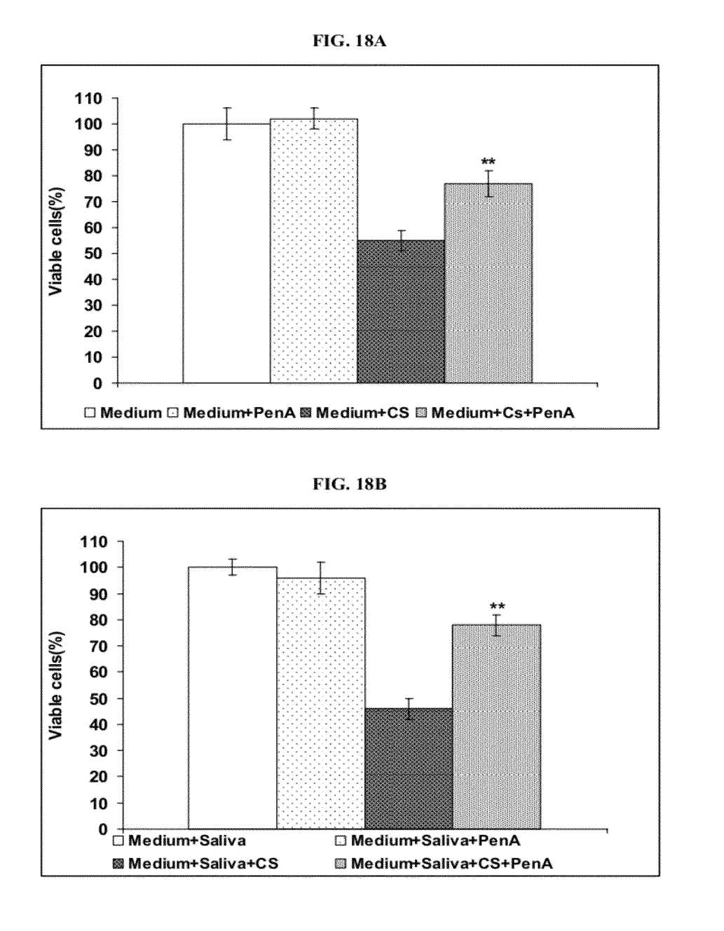

Similar results were obtained with oral cancer cells (SCC-25). As shown in FIG. 18A, exposure of oral cancer cells (SCC-25) to CS per se (in the absence of saliva) for 120 minutes resulted in a survival loss of 45% (p<0.01). This was substantially prevented by addition of 5 mM PenA to the incubation medium prior to the exposure of the cells to CS, which resulted in a rather limited survival loss, of 23% only (p=0.0024). Similar phenomenon was demonstrated when saliva was added to the incubation medium prior to the exposure to CS (FIG. 18B). Under these conditions the lethal synergistic effect of saliva and CS was demonstrated and the induced survival loss was of 54% (p<0.01). In this case addition of 5 mM PenA also substantially prevented this induced loss of survival, resulting in a limited survival loss of 22% only (p=0.015) (FIG. 18B).

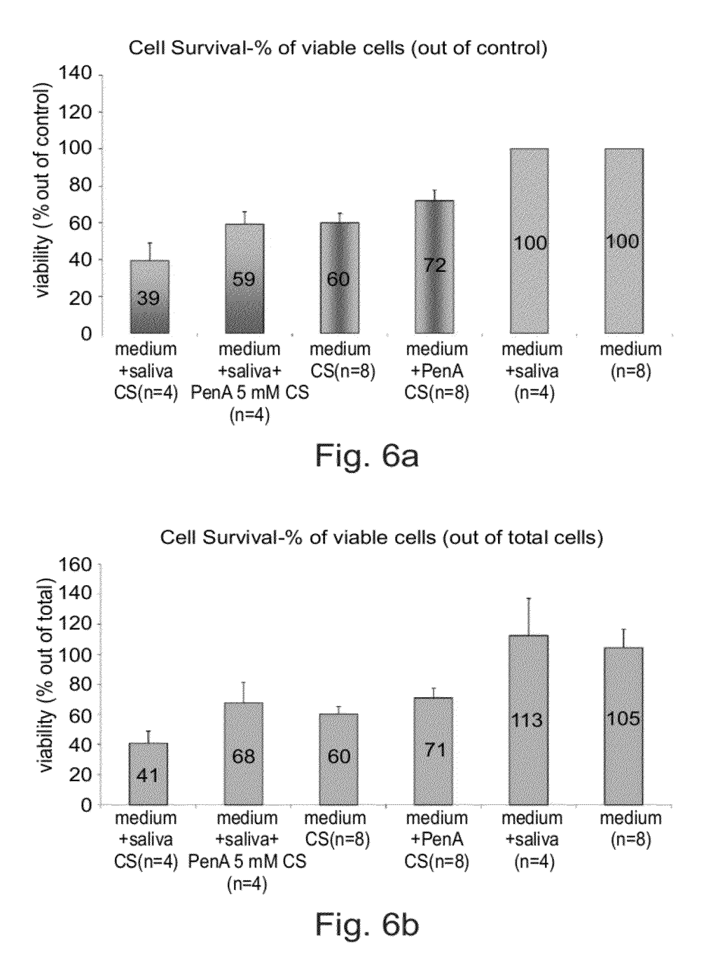

FIGS. 6A and 6B present the percent of viable H1299 cells out of control (FIG. 6A) and out of total number of cells (FIG. 6B) upon incubation in medium alone or in medium supplemented with 30% (v/v) saliva, with or without prior addition of PenA to the medium, and with or without exposure to CS, for 120 minutes. These data clearly demonstrate the drastic effect of PenA on cell survival, with a 20% increase of cell survival in cells incubated in saliva-containing medium and a 12% increase of cells survival in cells incubated in medium alone (See, FIG. 6A).

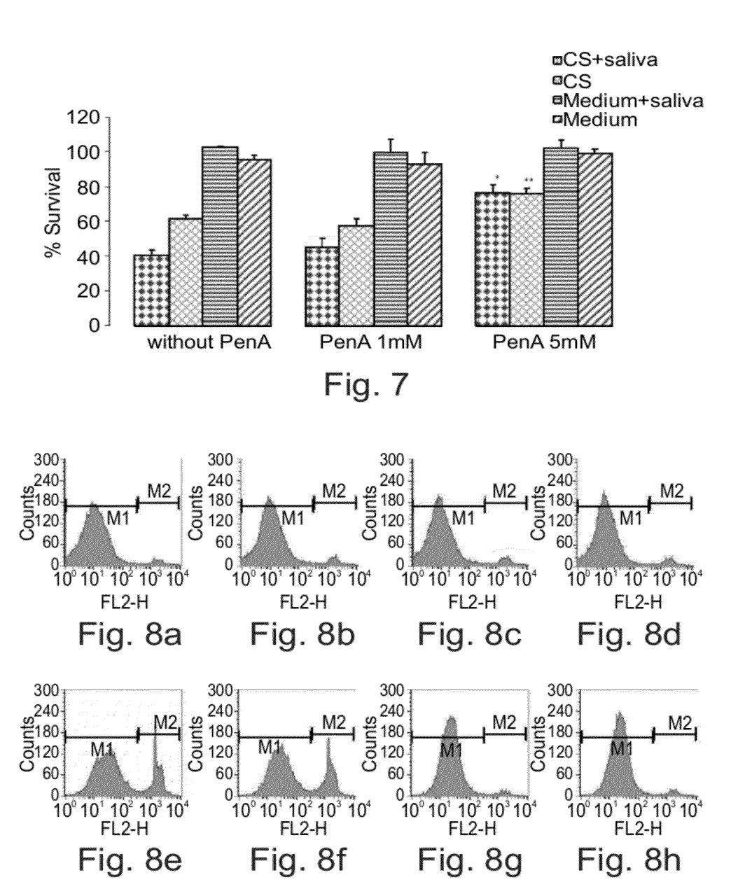

FIG. 7 presents the effect of 1 mM and 5 mM penicillamine (PenA) on the survival rate of H1299 cells incubated in medium and PenA or in medium containing 30% (v/v) saliva and PenA, and exposed to CS, compared with non-treated cells and further demonstrate a dose-dependent effect of penicillamine.

As shown in FIG. 7, the survival rate of cells that contained saliva was 40.4% following 120 minutes of CS exposure, while the addition of 5 mM PenA significantly raised the survival rate up to 76.4% (*p<0.001). The addition of 5 mM PenA also protected from the lethal effect of CS alone, and significantly raised the survival rate from 61.5% to 75.5% (**p<0.001). The unique protection phenomenon that prevent cells death, not only from the lethal synergism effect induced by CS and saliva, but also from the lethal effect of the CS itself, demonstrates a novel protective PenA mechanism.

In order to verify the interesting protective effect that PenA has on cells survival following exposure to CS with or without saliva, the fluorescent molecule Propidium Iodide (PI), which interchalates into double-stranded nucleic acids, was used. PI is excluded by viable cells, and penetrates to cell membranes of dead cells.

H1299 cells were incubated for 120 minutes with or without the presence of saliva in the culture medium and exposed to CS. Cells were also incubated in the presence of 5 mM PenA prior the exposure of CS.

Cell viability was evaluated by fluorescence assay. The results are presented in Table 1 below.

TABLE 1

| | Positive dead cells (%) | |
|---|---|---|
| CS + saliva | 23.32 ± | 0.36 |
| CS | 24.51 ± | 4.12 |
| CS + PenA + saliva | 2.33 ± | 0.29 * |
| CS + PenA | 2.98 ± | 0.06 ** |
| Medium + saliva | 3.21 ± | 0.42 |
| Medium | 4.12 ± | 1.02 |
| Medium + PenA + saliva | 4.15 ± | 0.56 |
| Medium + PenA | 3.75 ± | 0.81 |

Data are each expressed as the mean ± SD. (n = 4).
* P < 0.001,
** P < 0.005 vs without PenA.

FIG. 8 further presents the data obtained in these fluorescence studies. As shown in FIG. 8, cellular uptake of PI was increased after 120 minutes of CS exposure. As shown in FIG. 8 (panels E and F) and table 1, CS exposure caused a significant decrease of cell viability (PI-positive cells) both in the presence (23.32%±0.36%) and in the absence (24.515±4.12%) of saliva. The presence of saliva in the cultured medium did not lead to additional cellular loss. The addition of PenA to the cultured medium completely protected from cell death after 120 minutes of CS exposure with (2.33%±0.29%) or without (2.98%±0.06%) the presence of saliva, in a very significant manner.

Without being bound to any particular theory, it is noted that while PenA is known as a copper chelator, these results may suggest that when no chelation of the redox active copper was effected prior to the exposure of the cells to CS, the rate of cells killing was doubled. Since PenA did not alter the CS-induced cell death in the absence of saliva, it may be suggested that the redox active copper ions originate in the saliva and not in the CS. However, it may further be suggested, without being bound to any particular theory, that PenA reacts as an anti-inflammatory agent and exerts its beneficial activity via modulation of the innate immune activity and hence can be utilized for ameliorating tobacco-associated damage to tissues other than the aerodigestive tract.

Modification of CS and/or Saliva Effects by GSH:

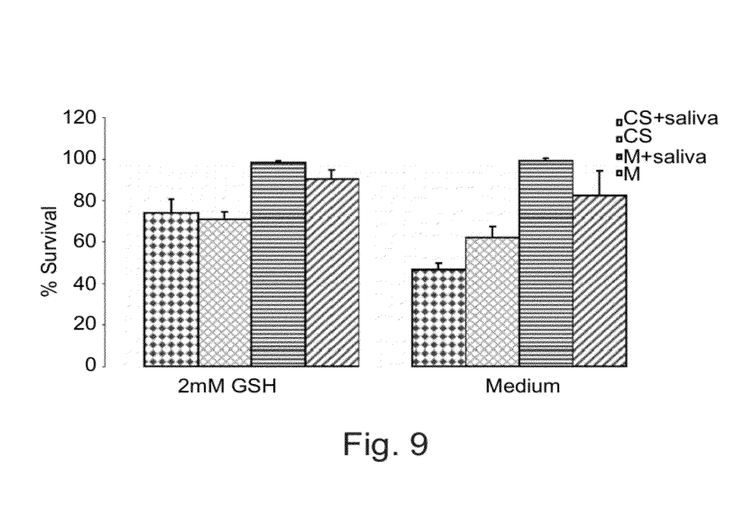

FIG. 9 presents the percents of viable H1299 cells upon incubation in medium alone or in medium supplemented with 30% (v/v) saliva, with or without prior addition of 2 mM GSH to the medium, and with or without exposure to CS. These data clearly demonstrate the drastic effect of GSH on cell survival, with more than 50% increase of cell survival in cells incubated in saliva-containing medium following GSH treatment.

Glutathione (GSH) is a tripeptide (L-c-glutamyl-L-cysteinyl-glycine) containing a thiol group. GSH is an important protective antioxidant against free radicals and other oxidants, and has been implicated in immune modulation and inflammatory responses. Glutathione exists in reduced (GSH) and oxidized (GSSG) states. In the reduced state, the thiol group of cysteine is able to donate a reducing equivalent (H⁺+e⁻) to other unstable molecules, such as reactive oxygen species. In donating an electron, glutathione itself becomes reactive, but readily reacts with another reactive glutathione to form glutathione disulfide (GSSG). GSH protects cells against CS-born aldehydes, which are known to mediate CS damage.

Addition of 2 mM GSH to the cultured medium inhibited the lethal synergistic effect and increased the survival rate, while there was no additional protection following CS exposure alone. These results demonstrate that aldehydes lethal effect can be accelerate by saliva and CS exposure. It can be assumed that the lack of GSH protection following CS alone is a result of cell-type specific that dose not susceptible to low active-aldehydes.

The possible protection ability of GSH is also demonstrated by the ability to prevent the rapid downregulation of p53 following CS and saliva exposure. This effect may be attributes to the decrease of oxidized thiols by GSH, resulted in less p53 aggregation and subsequent degradation.

Effect of Saliva and Cigarette Smoke, with and without PenA, on Protein Carbonylation Level of H1299 Cells:

Protein carbonylation is a covalent modification of a protein, induced by reactive oxygen intermediates or by-products of oxidative stress, and is the most general and well-used biomarker for severe oxidative protein damage.

In order to evaluate whether the synergistic effect of CS and saliva increases cellular oxidative stress and whether PenA act as antioxidant agent, carbonyl groups formation using DNPH was quantified on H1299 cells exposed during 90 minutes to CS and supplemented with saliva. Samples were also incubated with 5 mM PenA prior the exposure of CS.

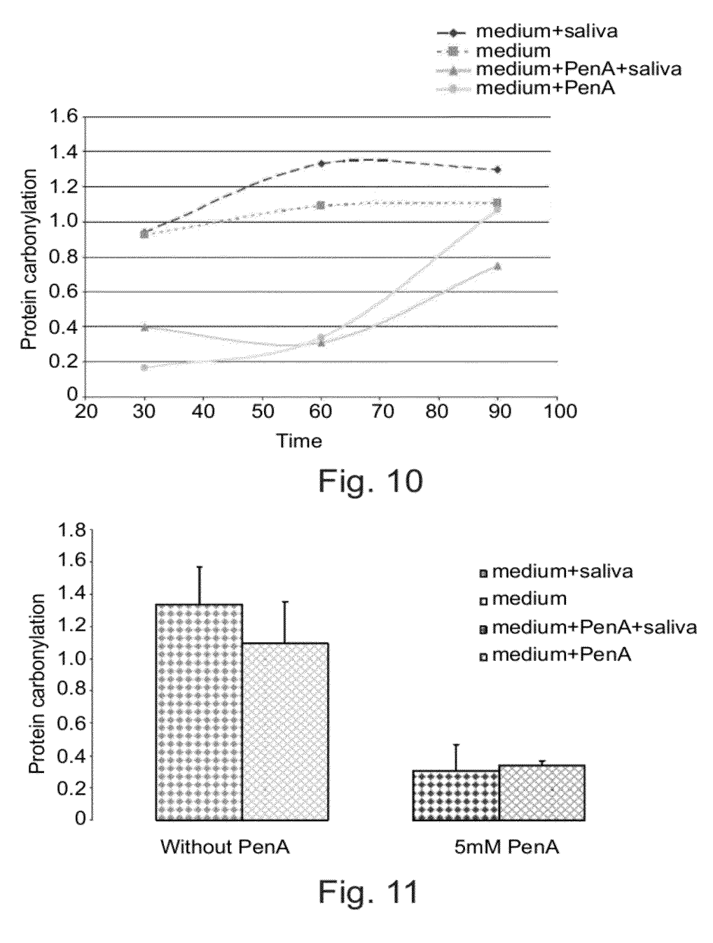

FIG. 10 presents the time-dependent effect of CS on protein carbonylation and clearly demonstrates a sharp increase in protein carbonylation within the first hour of exposure to CS, in cells incubated with saliva-containing medium. A moderate increase is observed in cells incubated with medium alone, thus further demonstrating the synergistic effect of saliva and CS on tobacco-associated damage.

As shown in FIG. 10, during 30, 60 and 90 minutes, there was 7.5, 6.9 and 6.7-fold increasing in the protein carbonyl content following CS exposure, respectively. The addition of saliva to the cultured medium resulted in significant increase in the protein carbonyl content. Thus, at 30, 60 and 90 minutes following the exposure of CS and saliva there were 6.5, 19 and 16-fold increase in the protein carbonyl content, respectively.

As shown in FIG. 11, the presence of PenA resulted in a significant decrease of proteins carbonyl content. These results show a 3.2-fold decrease in the protein carbonyl content on cells that were exposed to CS during 60 minutes, as compared to measurements without the presence of PenA (4.45 nmol/mg protein versus 14.6 nmol/mg protein, p<0.01). In addition, samples that were exposed to CS and saliva demonstrated a 3.6-fold decrease in proteins carbonyl content following the addition of PenA (4.02 nmol/mg protein versus 14.6 nmol/mg protein without PenA, p<0.01).

Effect of Desferal (DES) on Cells Viability:

In order to verify the possible role of redox iron on the synergistic effect of CS and saliva, the potent iron chelator DES was added to cells prior to exposing the cells to CS.

H1299 cells were incubated for 120 minutes with or without the presence of saliva in the culture medium and exposed to CS. Cells were also incubated in the presence of 5 mM DES prior the exposure of CS. Cell viability was evaluated by Trypan blue exclusion dye assay.

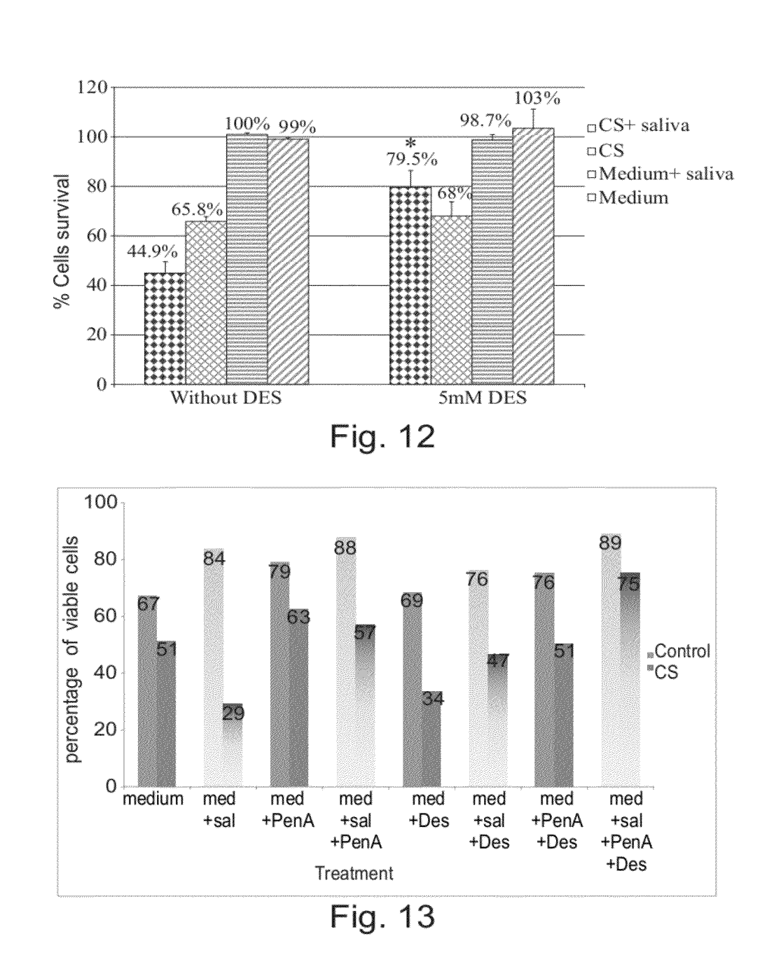

As shown in FIG. 12, DES significantly protected the cells from the lethal synergistic effect of saliva and CS. While the cellular death rate with no protection was of 55.1%, it dropped to 20.5% following the addition of 5 mM DES (p<0.005). No significant change on the lethal effect of CS on the lung cancer cells was observed in the absence of saliva.

Synergistic Effect of Desferal and PenA on Cell Viability:

H1299 cells were incubated for 120 minutes with or without the presence of saliva in the culture medium and exposed to CS. Cells were also incubated in the presence of 5 mM DES, 5 mM PenA and a mixture of DES and PenA, prior the exposure of CS. Cell viability was evaluated by Trypan blue exclusion dye assay.

As shown in FIG. 13, a synergistic effect was demonstrated for a combination of DES and PenA, particularly in cells incubated in the presence of saliva and exposed CS.

Mitochondrial Potential (ΔΨₘ):

Mitochondrial membrane permeabilization is an essential step leading to apoptosis. Disruption of ΔΨₘ irreversibly commits cells to undergo death and is an early marker of apoptosis. Furthermore, cigarette smoke was previously shown to induce mitochondrial depolarization in human monocytes. Therefore, reduced ΔΨₘ, as measured by diminished incorporation of the fluorescent dye JC-1, was used as an early indicator for CS-induced apoptosis.

H1299 lung cancer calls were exposed for 120 minutes to CS with or without saliva. Samples were also incubated in the presence of 5 mM PenA or 5 mM DES prior the exposure of CS. JC-1 incorporation was measured by flow cytometry method.

As shown in FIG. 14, CS induced a significant drop in ΔΨₘ. The addition of 33% saliva did not enhanced further loss of ΔΨₘ, thus showing that ΔΨₘ cannot demonstrate the synergistic effect between CS exposure and saliva (42.5% versus 39.85% in the presence of saliva). The addition of 5 mM PenA to H1299 lung cancer cells prior the exposure of CS resulted in a strong protection against ΔΨₘ losses. However, no significant change was measured following the addition of 33% saliva. The addition of 5 mM DES was ineffective in protecting from ΔΨₘ losses following CS and saliva exposure, while it had a moderate protection against of ΔΨₘ following CS exposure alone.

Mitochondrial Oxidative Damage:

The mitochondrial quality is the amount of all the intra-cellular mitochondria and their content, and it is an important index of mitochondrial injury. Nonyl-Acridine Orange (NAO) is a fluorescent dye that can bind specifically to unoxidizable cardiolipin (CL), a mitochondrial phospholipid, independent of the mitochondria energetic state (ΔΨₘ). NAO lost its affinity for CL with a hydroperoxide fatty acid (CL-OOH).

NAO flow cytometry was used to measure the quality of H1299 lung cells exposed for 120 minutes to CS with or without saliva. The potential metal chelators PenA and DES were evaluated for their protection from CL oxidation following CS exposure.

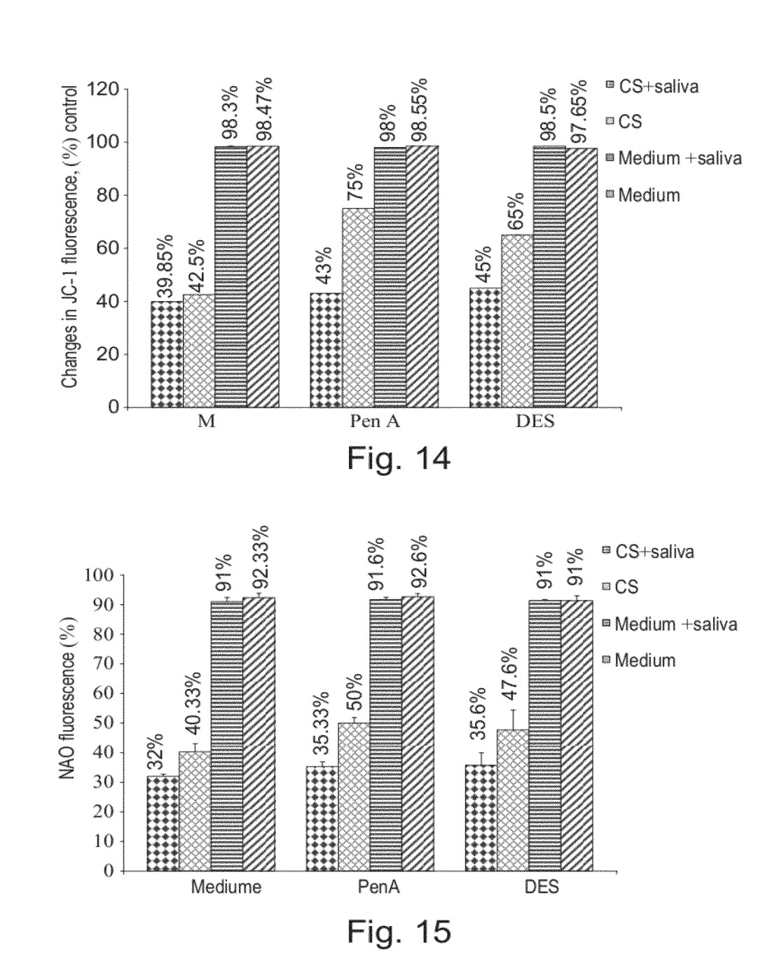

As shown in FIG. 15, the extent of binding of NAO to mitochondrial CL following 120 minutes of CS exposure, with or without the addition of 33% saliva to cells medium, was significantly lower than the controls group. Additional decrease in the extent of staining with NAO was observed in cells that were exposed to CS and 33% saliva in a moderate signification (40.3% versus 32% with the addition of saliva, P=0.01). The amount of NAO bound to mitochondrial CL following 120 minutes of CS exposure was the same irrespective of the presence of potent antioxidants such as DES or PenA.

Total p53 Expression Levels:

It is known that some oxidative stresses such as CS exposure can damage DNA and induce cell cycle arrest via ATM-p53-p21 pathway. The downstream target, a retinoblastoma protein (pRb) can be activated (hypophosphorylated) through induction of cyclin-dependent kinase inhibitors (CDKIs), such as p21, a transcriptional target of p53, or p16 in response to DNA damage (see introduction).

The effect of CS on p53 accumulation was analyzed. Human lung cancer cells were exposed for 120 minutes to CS with or without the presence of saliva. Samples were also incubated with 5 mM PenA or 2 mM GSH prior the exposure. Western blot analysis was performed and the amount of p53 was evaluated.

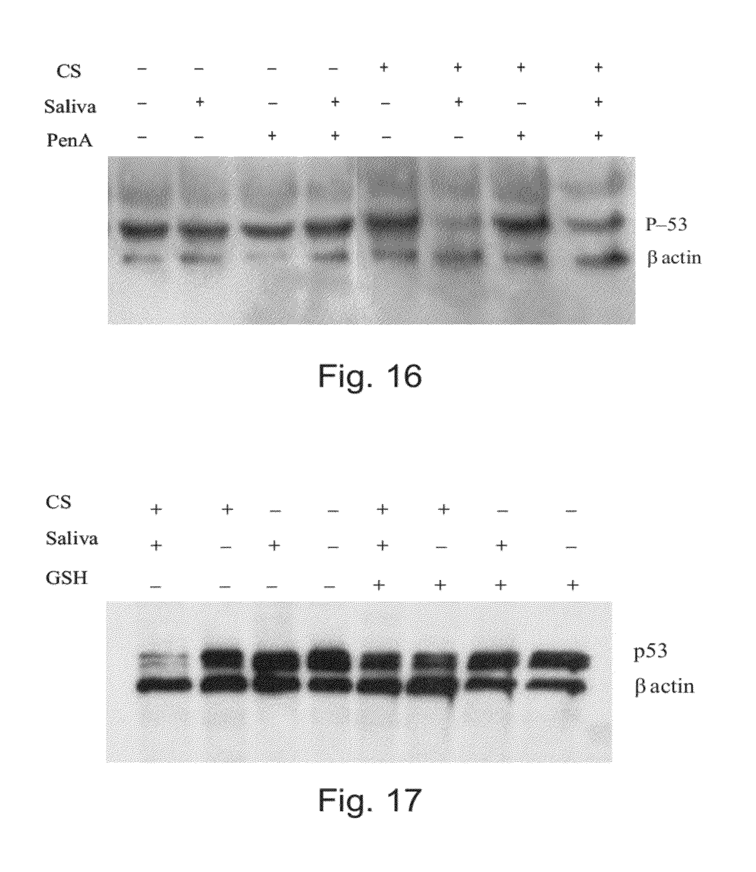

As shown in FIG. 16, exposure to CS did not alter p53 protein level. Supplement of saliva to the cultured medium induced a dramatically decrease in p53. Addition of 5 mM PenA prior the exposure of CS and saliva, partially prevented the dramatically decrease in p53 expression. The antioxidant GSH reveled a stronger protection pattern by totally preventing the p53 downregulation following exposure of CS and 33% saliva, as shown in FIG. 17.

Mechanistic Insights:

The above described studies show that PenA was found to be a novel antioxidant agent. Addition of PenA to the cultured medium prior the exposure to CS resulted in a significant protection against cellular loss and protein modification, irrespective to the presence of saliva.

PenA was found to exhibit a protective effect in the two studies cell lines, representing two most devastating cancers of the aero-digestive tract: lung cancer and oral cancer.

Notably, a synergistic effect is demonstrated on cell viability following incubation of H1299 with both DES and PenA.

As discussed in the Background section hereinabove, two mechanisms that are related to oral cancer are based on: (i) the injurious effects of salivary redox active iron ions, which turn low reactive free radicals in the CS into the most aggressive and highly reactive radicals existing—hydroxyl radicals; and (ii) the direct effects of CS inborn aldehydes. Aldehydes effects also play a role in lung carcinogenesis [Smith et al., Inhal Toxicol. 2006 18(9):667-77; Feng wt al., Proc Natl. Acad. Sci. USA, 2006 103(42):15404-9]. Thus, the effects of salivary iron ions is involved in the initiation, promotion and progression of cancer at the epithelial regions being constantly bathed by saliva while interacting with CS (oral cavity and down to the larynx) and the effect of aldehydes takes place all along the aero-digestive tract, down through the trachea to the lungs.

The data presented herein clearly show that both DES and GSH did not protect the cell death induced by CS per se, though they did protect against the cell death induced by the synergistic effect of CS and saliva. Without being bound by any particular theory, it is suggested that DES and GSH act via chelating of salivary redox active iron by the DES on one hand, and detoxifying CS-inborn aldehydes by the GSH on the other hand.

PenA was shown to exhibit even a more pronounced protective effect, and was further shown to prevent cell death even in the absence of saliva, and moreover, to protect against CS-induced decrease in p53 level of expression.

The novel findings presented herein are used to understand aspects related to the mechanistic and therapeutic levels associated with CS-induced pathologies, such as cancer.

Without being bound to any particular theory, it is suggested that PenA exerts its protective effect via the following possible mechanisms:

As a copper chelator for free copper ions that are presents in saliva. Neutralization of these redox active copper ions prevents their participation in Fenton and Haber-Wiss reaction and transform into highly reactive free radicals. This mechanism can explain the increase of cells survival and the decrease of proteins carbonylation following CS and saliva exposure;

As a copper chelator for free copper ions that originate from CS and from denaturative proteins that use copper as a cofactor (such as chaperones and SOD1). Following CS exposure, several proteins are being oxidatively damaged, resulting in a cellular copper resource release. PenA may also neutralize these copper resources and therefore defense CS direct damage that is non-salivary mediated;

As an inhibitor of the transcription factor AP-1. Oxidants in cigarette smoke can activate the mitogen-activated protein kinase (MAPK) signaling cascades in lung epithelial cells in-vitro and in-vivo. These signaling pathways lead to the enhanced ability of Jun and Fos family members (components of the AP-1) to activate transcription of a number of AP-1 dependent target genes involved in cell proliferation, death and inflammation. PenA can block the binding of AP-1 to the DNA and therefore may prevent apoptosis and inflammatory reaction. This mechanism may explain the decrease in cellular death following CS exposure. This possible mechanism of PenA may have therapeutic impact on chronic lung inflammation in heavy smokers;

As an antioxidant that may scavenge some of the free radicals that originate from CS and enhanced by the presence of saliva. Thus, decrease of free radicals level can result in less cellular death and protein oxidation.

PenA was also found to prevent ΔΨₘ depletion on cells that exposed to CS per se but not samples that exposed to CS and saliva. Thus, PenA protects from CS direct damage irrespective of saliva.

PenA is also capable of preventing p53 downregulation following CS and saliva exposure. This effect may be attributed to CS salivary mediate damage while addition of PenA partially prevented this effect and thus, stabilized p53 and prevents its degradation.

The data presented herein suggest that removing copper ions which may originate in saliva, pleural fluid, CS or degraded and oxidized proteins, may be of importance in preventing aero-digestive cancers. Both iron and copper are known to be redox-active in the saliva and as such have been implemented in the pathogenesis of oral cancer [Reznick et al., Br J Cancer 2004; 91:111-118; Hasnis et al., Int. J. of Biochemistry and Cell Biology 36(5), 826-839, (2004)].

The data presented herein suggests a similar role for redox-active metal ions in the pathogenesis of lung cancer. Accordingly, chelating agents of iron and/or copper and/or any other ions of metal that can be in a redox-active state, namely, can assume more than one oxidation sate other than 0, can be used in treating or preventing lung cancer. P53 is a major anti-carcinogenic and pro-apoptotic gene which protects the cell from DNA damage by slowing down cell replication via G1 arrest and/or by inducing apoptosis. It is established that oxidative stress and CS can damage DNA and induce cell cycle arrest via ATM-p53-p21 pathway [Helt et al., Toxicol Sci. 2001 October; 63(2):214-22]. The protective effect of PenA presented herein may suggest that redox active ions such as iron and copper in the pleural fluid play a carcinogenic role related to CS and induction of lung cancer. Thus, the CS-induced reduced p53 expression might lead to the activation of growth-promotive pathways, as well as to inactivation of growth-inhibitory factors, such as the cyclin-dependent kinase inhibitor p21, and thus to induction of cancer. The fact that GSH also protected against decrease in p53 level of expression suggests that CS aldehydes are potential injurious agents in this regard and that the effect exhibited by PenA may result from its potent anti-aldehyde activity. This protective effect may be attributed to the decrease of oxidized thiols by GSH, resulting in a reduced p53 aggregation and a subsequent degradation.

The observation regarding CS-induced decrease in p53 level of expression in the presence of saliva, and its prevention by PenA and other metal chelators may explain the carcinogenesis of CS-induced cancers (such as lung and oral cancers) which are often characterized by malfunctioning p53. Since redox-active iron and copper in the pleural fluid and in the saliva, when encountered with CS, may be responsible for carcinogenesis mediated via altering p53 function, chelation of redox-active metals are suggested herein as an efficient tool for prevention of CS-induced cancers.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An article of manufacturing comprising tobacco and/or a tobacco packaging material, wherein at least a portion of said tobacco and/or tobacco packaging material comprises an agent selected from the group consisting of penicillamine and a structural analog of penicillamine.

2. The article of manufacturing of claim 1, wherein said at least a portion of said tobacco and/or said tobacco packaging material is in contact with an aerodigestive tract of a subject using the article of manufacturing.

3. The article of manufacturing of claim 2, wherein said tobacco packaging material is a filter, and said filter is designed and configured so as to enable release of said agent therefrom when in use by the subject.

4. The article of manufacturing of claim 1, wherein said agent is penicillamine.

5. The article of manufacturing of claim 4, said agent is D-penicillamine.

6. The article of manufacturing of claim 1, wherein said at least a portion of said tobacco and/or said tobacco packaging material further comprises at least one additional agent capable of reducing or preventing tobacco smoke-associated damage in a subject using the article of manufacturing.

7. The article of manufacturing of claim 6, wherein said agent is desferal.

8. A method of treating or preventing a tobacco-associated damage in a subject in need thereof, the method comprising administering to the subject a therapeutically effective amount of an agent selected from the group consisting of penicillamine and a structural analog thereof, thereby treating or preventing the tobacco-associated damage.

9. The method of claim 8, further comprising administering to said subject at least one additional agent that is capable of reducing or preventing the tobacco-associated damage.

10. The method of claim 9, wherein said additional agent is an antioxidant.

11. The method of claim 10, wherein said antioxidant is desferal.

12. A pharmaceutical composition comprising an agent selected from the group consisting of penicillamine and a structural analog thereof and a pharmaceutically acceptable carrier, the composition being packaged in a packaging material and identified in print, in or on said packaging material, for use in the treatment of a tobacco-associated damage.

13. The pharmaceutical composition of claim 12, further comprising at least one additional agent that is capable of reducing or preventing the tobacco-associated damage.

14. The pharmaceutical composition of claim 12, being in the form of a toothpaste, powder, liquid dentifrice, mouthwash, denture cleanser, chewing gum, lozenge, paste, gel or candy.
